(19) United States
(12) Patent Application Publication
(10) Pub. No.: US 2024/0058320 A1
(43) Pub. Date: Feb. 22, 2024

(54) MYDRIATIC COMPOSITIONS AND METHODS FOR FABRICATING THEREOF

(71) Applicant: Harrow IP, LLC, Nashville, TN (US)

(72) Inventor: DENNIS SAADEH, (US)

(21) Appl. No.: 18/266,491

(22) PCT Filed: Dec. 9, 2021
(86) PCT No.: PCT/US2021/062643

§ 371 Date: Jun. 9, 2023

Related U.S. Application Data
(60) Provisional application No. 63/127,087, filed on Dec. 17, 2020.

(51) Int. Cl. A61K 31/4709; A61K 31/137; A61K 31/407; A61K 31/196; A61K 31/4409; A61K 47/38; A61K 47/10; A61K 47/18; A61K 31/167

(52) U.S. Cl. CPC A61K 31/4709; A61K 31/137; A61K 31/407; A61K 31/196; A61K 31/4409; A61K 47/38; A61K 47/10; A61K 47/183; A61K 31/167

(57) ABSTRACT

Pharmaceutical compositions for intraocular injection and for topical administration are described, the compositions comprise therapeutically effective quantity of at least two mydriatic compounds, a non-steroid anti-inflammatory drug, and further, optionally, a gel forming compound, an anesthetic, an antibiotic, and a metal chelator. Methods for fabricating the compositions and using them are also described.

### CROSS-REFERENCE TO RELATED APPLICATIONS

This application claims priority to U.S. Provisional Application No. 63/127,087 filed Dec. 17, 2020, the entirety of which is incorporated herein by reference.

### FIELD OF THE INVENTION

The present invention relates generally to the field of ophthalmology and more specifically to injectable and topical ophthalmological mydriatic compositions and to methods of preparing such compositions.

### BACKGROUND

Epinephrine is a drug that is frequently used in ophthalmological treatments, procedures and surgeries, e.g., cataract surgery or glaucoma surgery to obtain the mydriatic effect that is often necessary. However, epinephrine quite easily gets oxidized causing the loss of its valuable medicinal properties. Therefore, most commercially available epinephrine contains preservatives and stabilizers, typically bisulfites, for a prolonged shelf life and stability.

However, using epinephrine with preservatives is undesirable as it can cause toxicity in the eye, putting patients at risk for toxic anterior segment syndrome (TASS), an acute inflammation of the anterior segment.

This disclosure provides alternative pharmaceutical compositions suitable for both intraocular injections and topical administration that can achieve such positive patient outcomes, and methods of fabricating and administering the same without drawbacks and deficiencies that characterize the use of epinephrine.

### SUMMARY

The present disclosure provides a pharmaceutical composition comprising a therapeutically effective quantity of at least two mydriatic compounds, such as phenylephrine and tropicamide; a therapeutically effective quantity of at least one non-steroid anti-inflammatory; optionally, a quantity of one or more gel forming compounds; and a pharmaceutically acceptable aqueous carrier. In some embodiments, the pharmaceutical composition is optionally free of preservatives and is optionally free of sulfites. In some embodiments, the pharmaceutical composition is formulated to be suitable for administration by an intraocular injection or for topical administration.

In some embodiments, the two mydriatic compounds are selected from the group consisting of phenylephrine, tropicamide, brimonidine, cyclopentolate, cyclopentolate hydrochloride, atropine, homatropine, scopolamine; and pharmaceutically acceptable salts thereof. In some embodiments, the two mydriatic compounds are phenylephrine and tropicamide, or pharmaceutically acceptable salts thereof.

In some embodiments, the non-steroid anti-inflammatory drug is selected from the group consisting of ketorolac, bromfenac, etodolac, sulindac, diclofenac, aceclofenac, nepafenac, tolmetin, indomethacin, nabumetone, ketoprofen, dexketoprofen, ibuprofen, flurbiprofen, dexibuprofen, fenoprofen, loxoprofen, oxaprozin, naproxen, aspirin, salicylic acid, diflunisal, salsalate, mefenamic acid, meclofenamic acid, flufenamic acid, tolfenamic acid, meloxicam, piroxicam, ternoxicam, droxicam, lornoxicam, isoxicam, celecoxib, rofecoxib, valdecoxib, parecoxib, lumiracoxib, etoricoxib, firocoxib, nimesulide, clonixin, licofelone, and pharmaceutically acceptable salts thereof. In some embodiments, the NSAID is ketorolac, or a pharmaceutically acceptable salt thereof. In some embodiments, the NSAID is diclofenac, or a pharmaceutically acceptable salt thereof.

In some embodiments, the one or more gel forming compounds is selected from the group consisting of alginic acid, sodium alginate, potassium alginate, calcium alginate, agar-agar, pectin, guar gum, xanthan gum, gelatin, poly(oxyethylene-co-oxypropylene) block copolymers, poly(N-isopropylacrylamide), poly(N-isopropylacrylamide-co-acrylic acid), poly(vinyl pyrrolidone), poly(4-vinylpyridine-co-ethylacrylate) block copolymers, poly(N-isopropylacrylamide-co-butyl methacrylate-co-ethylene glycol) block copolymers, carboxymethyl cellulose hydroxyethyl cellulose, methyl cellulose, hydroxypropyl cellulose, hydroxypropyl methylcellulose, polyoxyethylene sorbitan monolaurates, polyoxyethylene sorbitan monopalmitates, polyoxyethylene sorbitan monostearates, and polyoxyethylene sorbitan monooleates. In some embodiments, the one or more gel forming compounds comprises sodium alginate. In some embodiments, the one or more gel forming compounds comprises a quantity of a poly(oxyethylene-co-oxypropylene) block copolymer. In some embodiments, the one or more gel forming compounds comprises hydroxypropyl methylcellulose.

In some embodiments, the pharmaceutical composition further comprises a therapeutically effective quantity of at least one anesthetic compound selected from the group consisting of lidocaine, tetracaine, proparacaine, procaine, dyclonine, chloroprocaine, and pharmaceutically acceptable salts thereof. In some embodiments, the anesthetic compound is lidocaine, or a pharmaceutically acceptable salt thereof.

In some embodiments, the pharmaceutical composition further comprises a therapeutically effective quantity of at least one antibiotic selected from the group consisting of moxifloxacin, gatifloxacin, teicoplanin, telavancin, decaplanin, ramoplanin ciprofloxacin, besifloxacin, levofloxacin, gentamicin, tobramycin and amikacin, and pharmaceutically acceptable salts thereof. In some embodiments, the antibiotic is moxifloxacin, or a pharmaceutically acceptable salt thereof.

In some embodiments, the pharmaceutical composition further comprises at least one metal chelator selected from the group consisting of ethylenediaminetetraacetic acid and pharmaceutically acceptable salts thereof. In some embodiments, the pharmaceutically acceptable salt of ethylenediaminetetraacetic acid is disodium edetate.

In some embodiments, the pharmaceutical composition is substantially free of preservatives. In some embodiments, the pharmaceutical composition is substantially free of sulfites.

The present disclosure also provides a pharmaceutical composition formulated to be suitable for administration by topical administration, comprising: phenylephrine, or a pharmaceutically acceptable salt thereof; tropicamide, or a pharmaceutically acceptable salt thereof; ketorolac, or a pharmaceutically acceptable salt thereof; one or more of a poly(oxyethylene-co-oxypropylene) block copolymer, hydroxypropyl methylcellulose, and sodium alginate; and a pharmaceutically acceptable aqueous carrier. In some embodiments, the pharmaceutical composition is substantially free of preservatives and substantially free of sulfites. In some embodiments, the pharmaceutical composition further comprising one or more of: moxifloxacin, or a pharmaceutically acceptable salt thereof; EDTA, or a pharmaceutically acceptable salt thereof; boric acid, glycerin; polysorbate 80; sodium metabisulfite; and benzalkonium chloride.

In some embodiments, the pharmaceutical composition comprises: about 1.8 to about 2.8 wt % phenylephrine, or a pharmaceutically acceptable salt thereof; about 0.4 to about 1.4 wt % tropicamide; about 0.1 to about 0.8 wt % ketorolac, or a pharmaceutically acceptable salt thereof; about 0.1 to about 0.4 wt % of a poly(oxyethylene-co-oxypropylene) block copolymer; about 0.1 to about 0.4 wt % hydroxypropyl methylcellulose; about 0.1 to about 0.4 wt % sodium alginate; and a pharmaceutically acceptable aqueous carrier. In some embodiments, the pharmaceutical composition further comprises: about 0.1 to about 1.0 wt % moxifloxacin, or a pharmaceutically acceptable salt thereof; about 0.05 to about 0.15 wt % EDTA, or a pharmaceutically acceptable salt thereof; about 0.1 to about 0.8 wt % boric acid; about 0.25 to about 1.75 wt % glycerin; about 0.05 to about 0.5 wt % polysorbate 80; less than or equal to about 0.1 wt % benzalkonium chloride; and less than or equal to about 0.15 wt % sodium metabisulfite.

In some embodiments, the pharmaceutical composition comprises: about 2.3 wt % phenylephrine, or a pharmaceutically acceptable salt thereof; about 0.9 wt % tropicamide; about 0.37 wt % ketorolac, or a pharmaceutically acceptable salt thereof; about 0.4 wt % of a poly(oxyethylene-co-oxypropylene) block copolymer; about 0.23 wt % hydroxypropyl methylcellulose; about 0.23 wt % sodium alginate; and a pharmaceutically acceptable aqueous carrier. In some embodiments, the pharmaceutical composition further comprises: about 0.51 wt % moxifloxacin, or a pharmaceutically acceptable salt thereof; about 0.09 wt % EDTA, or a pharmaceutically acceptable salt thereof; about 0.3 wt % boric acid; about 1.2 wt % glycerin; about 0.2 wt % polysorbate 80; less than or equal to about 0.05 wt % benzalkonium chloride; and less than or equal to about 0.1 wt % sodium metabisulfite.

The present disclosure also provides a pharmaceutical composition formulated to be suitable for administration by intraocular injection, comprising phenylephrine, or a pharmaceutically acceptable salt thereof; tropicamide, or a pharmaceutically acceptable salt thereof; diclofenac, or a pharmaceutically acceptable salt thereof; and a pharmaceutically acceptable aqueous carrier. In some embodiments, the pharmaceutical composition is substantially free of preservatives and substantially free of sulfites. In some embodiments, the pharmaceutical composition further comprises one or more of: lidocaine, or a pharmaceutically acceptable salt thereof; EDTA, or a pharmaceutically acceptable salt thereof; boric acid, polysorbate 80; and sodium chloride.

In some embodiments, the pharmaceutical composition comprises: about 0.1 to about 0.5 wt % phenylephrine, or a pharmaceutically acceptable salt thereof; about 0.01 to about 0.03 wt % tropicamide; about 0.007 to about 0.013 wt % diclofenac, or a pharmaceutically acceptable salt thereof; and a pharmaceutically acceptable aqueous carrier. In some embodiments, the pharmaceutical composition further comprises: about 0.1 to about 2.5 wt % lidocaine; about 0.05 to about 0.15 wt % EDTA, or a pharmaceutically acceptable salt thereof; about 0.1 to about 0.8 wt % boric acid; about 0.05 to about 0.5 wt % polysorbate 80; and about 0.1 to about 0.8 wt % sodium chloride. In some embodiments, the pharmaceutical composition is preservative free. In some embodiments, the pharmaceutical composition is sulfite free.

In some embodiments, the pharmaceutical composition comprises: about 0.29 wt % phenylephrine, or a pharmaceutically acceptable salt thereof; about 0.02 wt % tropicamide; about 0.01 wt % diclofenac, or a pharmaceutically acceptable salt thereof; and a pharmaceutically acceptable aqueous carrier. In some embodiments, the pharmaceutical composition further comprises: about 0.98 wt % lidocaine; about 0.1 wt % EDTA, or a pharmaceutically acceptable salt thereof; about 0.3 wt % boric acid; about 0.1 wt % polysorbate 80; and about 0.38 wt % sodium chloride. In some embodiments, the pharmaceutical composition is preservative free. In some embodiments, the pharmaceutical composition is sulfite free.

The present disclosure also provides a method for surgically treating an ophthalmological disease, condition, disorder, syndrome or pathology in a mammalian subject in need of such treatment, the method comprising administering to the subject, during or prior to the surgical procedure, a pharmaceutically effective quantity of a pharmaceutical composition of the disclosure. In some embodiments, the ophthalmological disease, condition, disorder, syndrome or pathology is selected from the group consisting of cataract, glaucoma, diseases of retina, and floppy iris syndrome. In some embodiments, a gel forming compound is absent from the pharmaceutical composition, and the administration of the composition is by intraocular injection. In some embodiments, a gel forming compound is present in the pharmaceutical composition, and the administration of the composition is topical

### BRIEF DESCRIPTION OF THE DRAWINGS

FIG. 1A is graph depicting the stability over time of phenylephrine in the topical pharmaceutical composition described in Example 1.

FIG. 1B is graph depicting the stability over time of tropicamide in the topical pharmaceutical composition described in Example 1.

FIG. 1C is graph depicting the stability over time of ketorolac in the topical pharmaceutical composition described in Example 1.

FIG. 1D is graph depicting the stability over time of moxifloxacin in the topical pharmaceutical composition described in Example 1.

FIG. 2A is graph depicting the stability over time of phenylephrine in the injectable pharmaceutical composition described in Example 2.

FIG. 2B is graph depicting the stability over time of tropicamide in the injectable pharmaceutical composition described in Example 2.

FIG. 2C is graph depicting the stability over time of lidocaine in the injectable pharmaceutical composition described in Example 2.

FIG. 2D is graph depicting the stability over time of diclofenac in the injectable pharmaceutical composition described in Example 2.

### DETAILED DESCRIPTION

### A. Terms, Definitions and Abbreviations

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the invention, the present technology, or embodiments thereof, may alternatively be described using more limiting terms such as “consisting of or “consisting essentially of the recited ingredients.

The term “pharmaceutical composition” is defined as a chemical or biological compound or substance, or a mixture or combination of two or more such compounds or substances, intended for use in the medical diagnosis, cure, treatment, or prevention of disease or pathology.

The term “wt %” is defined as the weight percent of an identified component or set of components in a composition with respect to the total mass of the composition (including all other components and any carrier or vehicle that may be present, unless otherwise specified).

The term “preservative” for the purposes of the present invention refers to a chemical substance that is added to a pharmaceutical composition to prevent the pharmaceutical composition from deterioration, decomposition or degradation or to substantially reduce or decelerate the degree and/or the speed of such deterioration, decomposition or degradation.

Accordingly, “preservative-free” means a pharmaceutical composition that contains no more than trace amounts of a preservative, e.g., a pharmaceutical composition to which a preservative has not been added. Trace amounts of preservatives can include concentrations of about 1 nM or less, or about 0.01% of the pharmaceutical composition by weight or less or about 1 ng per dosage unit of pharmaceutical composition or less. In other embodiments, trace amounts of preservatives include concentrations of about 1 nM or less, about 100 pM or less, about 10 pM or less or about 1 pM or less; or about 0.01% or less, or about 0.001% or less or about 0.0001% or less, each of the pharmaceutical composition by weight. In other embodiments, trace amounts of preservatives in pharmaceutical compositions include pharmaceutical compositions wherein preservatives are present at about 1 ng or less, about 100 pg or less, about 10 pg or less or about 1 pg or less, each per dosage unit of pharmaceutical composition. It is explicitly understood that for the purposes of the present application, metal chelators such as EDTA defined below are not considered preservatives. Accordingly, compositions that comprise metal chelators such as EDTA are considered preservative-free if they include no other preservative(s).

In certain embodiments, pharmaceutical compositions of the disclosure may be “substantially free of preservatives.” As used herein, the phrase “substantially free of” and permutations thereof refers to a pharmaceutical composition of the disclosure containing a relatively low concentration of the preservative (e.g., a pharmaceutical composition that is formulated with a very small amount of preservative would be “substantially free of preservatives). By definition, a pharmaceutical composition that is “preservative free” is also “substantially free of preservatives”; however a compositions that is “substantially free of preservatives” may not necessarily be “preservative free.” Relatively low concentrations of preservatives include less than 1%, less than 0.5%, less than 0.25%, less than 0.15%, less than 0.1%, less than 0.05%, less than 0.01%, or less than 0.001% of the pharmaceutical composition by weight.

The term “anti-oxidant” for the purposes of the present invention refers to a chemical substance that is added to a pharmaceutical composition to prevent or inhibit the oxidation of compounds that are present in the composition. It is explicitly understood that for the purposes of the present application, anti-oxidants are not considered preservatives. Accordingly, compositions that optionally comprise anti-oxidants as described below are considered preservative-free if they include no other preservative(s).

The term “sulfite” refers to compounds that comprise the sulfite ion SO₃²⁻ such as normal salts of sulfurous acid H₂SO₃. For the purposes of the present application, the term “sulfite” is also inclusive of bisulfites, i.e., compounds that comprise the bisulfate ion HSO₃— such as acid salts of sulfurous acid.

Accordingly, “sulfite-free” means a pharmaceutical composition that contains no more than trace amounts of a sulfite, e.g., a pharmaceutical composition to which a sulfite has not been added. Trace amounts of sulfites can include relatively low concentrations of about 1 nM or less, or about 0.01% of the pharmaceutical composition by weight or less or about 1 ng per dosage unit of pharmaceutical composition or less. In other embodiments, trace amounts of sulfites include concentrations of about 1 nM or less, about 100 pM or less, about 10 pM or less or about 1 pM or less; or about 0.01% or less, or about 0.001% or less or about 0.0001% or less, each of the pharmaceutical composition by weight. In other embodiments, trace amounts of sulfites in pharmaceutical compositions include pharmaceutical compositions wherein preservatives are present at about 1 ng or less, about 100 pg or less, about 10 pg or less or about I pg or less, each per dosage unit of pharmaceutical composition. It is explicitly understood that for the purposes of the present application, metal chelators such as EDTA defined below are not considered sulfites. Accordingly, compositions that comprise metal chelators such as EDTA are considered sulfite-free if they include no other sulfite(s).

In certain embodiments, pharmaceutical compositions of the disclosure may be “substantially free of sulfites.” As used herein, the phrase “substantially free of” and permutations thereof refers to a pharmaceutical composition of the disclosure containing a relatively low concentration of sulfites (e.g., a pharmaceutical composition that is formulated with a very small amount of sulfites would be “substantially free of sulfites). By definition, a pharmaceutical composition that is “sulfite free” is also “substantially free of sulfites”; however a compositions that is “substantially free of sulfites” may not necessarily be “sulfite free.” Relatively low concentrations of sulfites include less than 1%, less than 0.5%, less than 0.25%, less than 0.15%, less than 0.1%, less than 0.05%, less than 0.01%, or less than 0.001% of the pharmaceutical composition by weight.

The term “phenylephrine” refers to any stereoisomer of 3-(1-hydroxy-2-(methylamino)ethyl)phenol. Preferably, the term “phenylephrine” refers to the R stereoisomer, shown below:

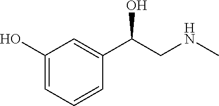

The term “tropicamide” refers to N-ethyl-3-hydroxy-2-phenyl-N-(pyridin-4-yl-methyl)propanamide, a chemical compound having the following chemical structure:

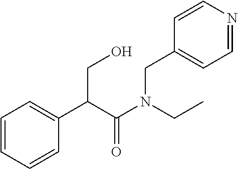 [SMILES: CCN(Cc1ccncc1)C(=O)C(CO)c1ccccc1]

The terms “non-steroid anti-inflammatory drug” or “NSAID” refer to substances or compounds that are free of steroid moieties and provide analgesic, antipyretic and/or anti-inflammatory effects.

The term “ketorolac” refers to 5-benzoyl-2,3-dihydro-1H-pyrrolizine-1-carboxylic acid, a chemical compound having the following chemical structure:

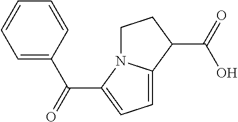 [SMILES: O=C(c1ccccc1)c1ccc2n1CCC2C(=O)O]

The term “diclofenac” refers to 2-(2-((2,6-dichlorophenyl)amino)phenyl)acetic acid, a chemical compound having the following chemical structure:

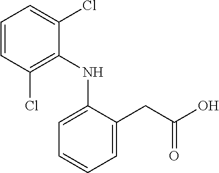 [SMILES: O=C(O)Cc1ccccc1Nc1c(Cl)cccc1Cl]

The terms “anti-bacterial” and “antibiotic” used herein interchangeably, refer to substances or compounds that destroy bacteria and/or inhibit the growth thereof via any mechanism or route.

The term “mydriatic” refers to substances or compounds that can causing dilation of the pupil of an eye.

The term “gel” refers to a solid three-dimensional network that spans the volume of a liquid medium and ensconces this liquid medium. A “gelling compound” or “gel forming compound” is defined as any compound that causes the formation of a gel or facilitates the formation of the same.

The term “anesthetic” refers herein to substances or compounds that induce temporary insensitivity to pain such as a temporary loss of sensation.

The term “lidocaine” refers to 2-diethylamino-N-(2,6-dimethylphenyl)acetamide, a chemical compound having the following chemical structure:

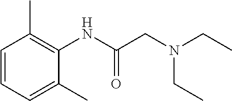 [SMILES: CCN(CC)CC(=O)Nc1c(C)cccc1C]

The abbreviation “EDTA” is 2,2′,2″,2′″-(ethane-1,2-diylbis(azanetriyl))tetraacetic acid (a chemical compound that is also known under several other names such as edetic acid or ethylenediaminetetraacetic acid), having the following chemical structure:

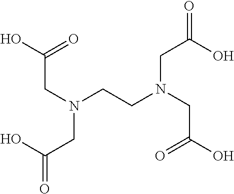 [SMILES: O=C(O)CN(CCN(CC(=O)O)CC(=O)O)CC(=O)O]

The terms “chelating agent” or “metal chelator” refer to a chemical compound that coordinates with a metal to form a chelate, which is a compound containing an organic ligand bonded to a central metal atom at two or more points.

The term “salt” refers to an ionic compound which is a product of the neutralization reaction of an acid and a base. Examples of salts (or salt forms) include, but are not limited to, mineral or organic acid salts of basic residues such as amines, alkali or organic salts of acidic residues such as carboxylic acids, and the like. Lists of suitable pharmaceutically acceptable salts are found in Remington's Pharmaceutical Sciences, 17th ed., Mack Publishing Company, Easton, Pa., 1985, p. 1418, the disclosure of which is hereby incorporated by reference in its entirety.

The term “block copolymer” refers to macromolecules that comprise two or more homopolymer subunits linked by covalent bond, having at least one structural feature (i.e., an intermediate non-repeating subunit) that is not present in the adjacent portions. For the purposes of the present specification, block copolymers are defined as both linear, branched and partially or fully cross-linked macromolecules.

The term “therapeutically effective amount” is defined as the amount of the compound or pharmaceutical composition that will elicit the biological or medical response of a tissue, system, animal or human that is being sought by the researcher, medical doctor or other clinician.

The term “pharmaceutically acceptable” refers to the property of a component (e.g., a carrier, whether diluent or excipient, or a salt form of an active ingredient) of a formulation to be compatible with the other ingredients of the formulation and not deleterious to the recipient thereof.

The terms “administration of a composition” or “administering a composition” is defined to include an act of providing a compound or pharmaceutical composition of the application to the subject in need of treatment.

As used herein, the term “treatment” or “treating,” is defined as the application or administration of a pharmaceutical composition, to a patient, or application or administration of a pharmaceutical composition to an isolated tissue or cell line from a patient (e.g., for diagnosis or ex vivo applications). Such treatments may be specifically tailored or modified, based on knowledge obtained from the field of pharmacogenomics.

The term “intraocular injection” refers to an injection that is administered by entering the eyeball of the patient.

The term “topical administration” refers to administering a compound or pharmaceutical composition locally to the skin or mucous membranes of a patient to treat various diseases or pathologies.

### B. Embodiments

Embodiments described in the present application are directed to pharmaceutical compositions that are free of the above-mentioned problems, drawbacks and defects. Specifically, the pharmaceutical compositions described herein exhibit high stability of the active ingredients comprised therein while being substantially preservative-free and substantially sulfite-free. Accordingly, the pharmaceutical compositions of the disclosure are capable of being stored for prolonged periods without oxidation or degradation of the active ingredients and can be administered to a subject without the risk of inducing toxic anterior segment syndrome or other forms of acute inflammation.

The pharmaceutical compositions of the disclosure comprise: a therapeutically effective quantity of at least two mydriatic compounds such as phenylephrine and tropicamide; a therapeutically effective quantity of at least one non-steroid anti-inflammatory drug (NSAID); optionally, a quantity of a gel forming compound; and a pharmaceutically acceptable carrier. Preferably, the pharmaceutical compositions of the disclosure are substantially free of preservatives and substantially free of sulfites. Further, the pharmaceutical compositions of the disclosure may be formulated to be suitable for administration by intraocular injection or for topical administration.

Mydriatic compounds for use in the pharmaceutical compositions of the disclosure include, by non-limiting example, atropine, brimonidine, cyclopentolate, homatropine, 4-hydroxyamphetamine, phenylephrine, scopolamine, tropicamide, and pharmaceutically acceptable salts thereof. In some embodiments, provided herein is a pharmaceutical composition comprising at least two mydriatic compounds, wherein one of the mydriatic compounds is phenylephrine, or a pharmaceutically acceptable salt thereof. In some embodiments, provided herein is a pharmaceutical composition comprising at least two mydriatic compounds, wherein one of the mydriatic compounds is phenylephrine hydrochloride. In some embodiments, provided herein is a pharmaceutical composition comprising at least two mydriatic compounds, wherein one of the mydriatic compounds is tropicamide, or a pharmaceutically acceptable salt thereof. In some embodiments, provided herein is a pharmaceutical composition comprising at least two mydriatic compounds, wherein the at least two mydriatic compounds are phenylephrine and tropicamide, or pharmaceutically acceptable salts thereof. In some embodiments, provided herein is a pharmaceutical composition comprising at least two mydriatic compounds, wherein the at least two mydriatic compounds are phenylephrine hydrochloride and tropicamide, or pharmaceutically acceptable salts thereof. Those having ordinary skill in the art may choose other mydriatic compounds, if desired.

Regardless of what additional components (whether additional active ingredient or excipient) are used as a part of the pharmaceutical composition, at least two mydriatic compounds are to be used as stated above.

The amount of the mydriatic compounds in the pharmaceutical compositions of the present disclosure may differ depending on whether the pharmaceutical composition is intended for topical application or for intraocular injections. For topical pharmaceutical compositions, the amount of the mydriatic compounds may, for example, be between about 0.01 and about 10.0 wt % of the total weight of the composition. Accordingly, in some embodiments, the pharmaceutical composition is formulated for topical administration and comprises mydriatic compounds in an amount between about 0.5 and about 5.0 wt % of the total weight of the composition. In other embodiments, the pharmaceutical composition is formulated for topical administration and comprises mydriatic compounds in an amount between about 1.5 and about 4.5 wt % of the total weight of the composition. In yet other embodiments, the pharmaceutical composition is formulated for topical administration and comprises mydriatic compounds in an amount of about 3.3 wt % of the total weight of the composition. In still further embodiments, the amount of the mydriatic compounds may, for example, be about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt % of the total weight of the composition. For the injectable pharmaceutical compositions, the amount of the mydriatic compounds may, for example, be between about 0.01 and about 3.0 wt %. Accordingly, in some embodiments, the pharmaceutical composition is formulated for injection and comprises mydriatic compounds in an amount between about 0.1 and about 1.0 wt % of the total weight of the composition; for example, about 0.1, 0.2, 0.3, 0.4, 0.5, 0.6, 0.7, 0.8, 0.9, or about 1.0 wt % of the total weight of the composition. In other embodiments, the pharmaceutical composition is formulated for injection and comprises mydriatic compounds in an amount between about 0.1 and about 0.5 wt % of the total weight of the composition; for example, about 0.1, 0.15, 0.2, 0.25, 0.3, 0.35, 0.4, 0.45, or about 0.5 wt % of the total weight of the composition. In yet other embodiments, the pharmaceutical composition is formulated for injection and comprises mydriatic compounds in an amount of about 0.3 wt % of the total weight of the composition.

Non-steroid anti-inflammatory drugs (NSAIDs) for use in the pharmaceutical compositions of the disclosure include NSAIDs typically used in the formulation of a pharmaceutical. Examples of NSAIDs for use in accordance with the disclosure include, but are not limited to, ketorolac, bromfenac, etodolac, sulindac, diclofenac, aceclofenac, nepafenac, tolmetin, indomethacin, nabumetone, ketoprofen, dexketoprofen, ibuprofen, flurbiprofen, dexibuprofen, fenoprofen, loxoprofen, oxaprozin, naproxen, aspirin, salicylic acid, diflunisal, salsalate, mefenamic acid, meclofenamic acid, flufenamic acid, tolfenamic acid, meloxicam, piroxicam, ternoxicam, droxicam, lornoxicam, isoxicam, celecoxib, rofecoxib, valdecoxib, parecoxib, lumiracoxib, etoricoxib, firocoxib, nimesulide, clonixin, licofelone, and pharmaceutically acceptable salts thereof. In some embodiments, the NSAID is ketorolac, or a pharmaceutically acceptable salt thereof. In some embodiments, the NSAID is ketorolac tromethamine. In some embodiments, the NSAID is diclofenac, or a pharmaceutically acceptable salt thereof. In some embodiments, the NSAID is diclofenac sodium. Those having ordinary skill in the art may choose other NSAIDs, if desired.

The amount of the NSAID(s) in the pharmaceutical compositions of the present disclosure may differ depending on whether the composition is intended for topical application or for intraocular injections. For topical compositions, the amount of the NSAID may, for example, be between about 0.01 and about 3.0 wt %. Accordingly, in some embodiments, the pharmaceutical composition is formulated for topical administration and comprises one or more NSAID in an amount between about 0.1 and about 1.5 wt % of the total weight of the composition. In other embodiments, the pharmaceutical composition is formulated for topical administration and comprises one or more NSAID in an amount between about 0.1 and about 0.8 wt % of the total weight of the composition. In still further embodiments the pharmaceutical composition is formulated for topical administration and comprises one or more NSAID in an amount of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, or about 3.0 wt % of the total weight of the composition. In yet other embodiments, the pharmaceutical composition is formulated for topical administration and comprises one or more NSAID in an amount of about 0.37 wt % of the total weight of the composition. For the injectable pharmaceutical compositions, the amount of the NSAID may, for example, be between about 0.001 and about 1.0 wt % of the composition. Accordingly, in some embodiments, the pharmaceutical composition is formulated for injection and comprises one or more NSAID in an amount between about 0.005 and about 0.5 wt % of the total weight of the composition. In other embodiments, the pharmaceutical composition is formulated for injection and comprises one or more NSAID in an amount between about 0.007 and about 0.013 wt % of the total weight of the composition. In still further embodiments, the pharmaceutical composition is formulated for injection and comprises one or more NSAID in an amount of about 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, or about 0.5 wt % of the total weight of the composition. In yet other embodiments, the pharmaceutical composition is formulated for injection and comprises one or more NSAID in an amount of about 0.01 wt % of the total weight of the composition.

When present, any suitable gel forming compounds can be used in the pharmaceutical compositions described herein. Examples of gel forming compounds for use in accordance with the disclosure include, but are not limited to, alginic acid, sodium alginate, potassium alginate, calcium alginate, agar-agar, pectin, guar gum, xanthan gum, gelatin, poly(oxyethylene-co-oxypropylene) block copolymers, poly(N-isopropylacrylamide), poly(N-isopropylacrylamide-co-acrylic acid), poly(vinyl pyrrolidone), poly(4-vinylpyridine-co-ethylacrylate) block copolymers, poly(N-isopropylacrylamide-co-butyl methacrylate-co-ethylene glycol) block copolymers, carboxymethyl cellulose, hydroxyethyl cellulose, methyl cellulose, hydroxypropyl cellulose, hydroxypropyl methylcellulose, polyoxyethylene sorbitan monolaurates, polyoxyethylene sorbitan monopalmitates, polyoxyethylene sorbitan monostearates, polyoxyethylene sorbitan monooleates, and combinations thereof. In some embodiments, the gel forming compound is guar gum. In some embodiments, the gel forming compound is xanthan gum. In some embodiments, the gel forming compound is a poly(oxyethylene-co-oxypropylene) block copolymer. In some embodiments, the gel forming compound is POLOXAMER® 407. In some embodiments, the gel forming compound is hydroxypropyl methylcellulose. In some embodiments, the gel forming compound is METHOCEL® E4M. In some embodiments, the gel forming compound is sodium alginate. In some embodiments, disclosed herein is a pharmaceutical composition comprising at least one gel forming compound selected from: a poly(oxyethylene-co-oxypropylene) block copolymer, hydroxypropyl methylcellulose, and sodium alginate. In some embodiments, disclosed herein is a pharmaceutical composition comprising a poly(oxyethylene-co-oxypropylene) block copolymer, hydroxypropyl methylcellulose, and sodium alginate. In some embodiments, disclosed herein is a pharmaceutical composition comprising POLOXAMER® 407, METHOCEL® E4M, and sodium alginate. Those having ordinary skill in the art may choose other gel forming compounds, if desired.

Only pharmaceutical compositions formulated for topical application may include one or more gel forming compounds. Gel forming compounds are not to be used in pharmaceutical compositions formulated for intraocular injection. When present, the amount of the gel forming compound(s) present in a pharmaceutical composition for topical application may, for example, be between about 0.01 and about 7.0 wt %. Accordingly, in some embodiments, the pharmaceutical composition is formulated for topical administration and comprises one or more gel forming compound in an amount between about 0.1 and about 3.0 wt % of the total weight of the composition. In additional embodiments, the pharmaceutical composition is formulated for topical administration and comprises one or more gel forming compound in an amount of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, or about 7.0 wt % of the total weight of the composition. In other embodiments, the pharmaceutical composition is formulated for topical administration and comprises one or more gel forming compound in an amount between about 0.25 and about 1.25 wt % of the total weight of the composition. In yet other embodiments, the pharmaceutical composition is formulated for topical administration and comprises one or more gel compound in an amount of about 0.65 wt % of the total weight of the composition.

The pharmaceutical compositions of the disclosure can also include one or more antibiotic typically used in the formulation of a pharmaceutical. Examples of antibiotics for use in accordance with the disclosure include, but are not limited to, moxifloxacin, gatifloxacin, teicoplanin, telavancin, decaplanin, ramoplanin, gentamicin, tobramycin, levofloxacin, besifloxacin, ciprofloxacin, am ikacin, and pharmaceutically acceptable salts thereof. In some embodiments, disclosed herein is a pharmaceutical composition comprising an antibiotic, wherein the antibiotic is moxifloxacin, or a pharmaceutically acceptable salt thereof. In some embodiments, disclosed herein is a pharmaceutical composition comprising an antibiotic, wherein the antibiotic is moxifloxacin hydrochloride monohydrate. Those having ordinary skill in the art may choose other antibiotics, if desired.

When present, the amount of the antibiotic(s) in the pharmaceutical compositions of the present disclosure may differ depending on whether the composition is intended for topical application or for intraocular injections. For topical compositions, the amount of the antibiotic may, for example, be between about 0.01 and about 5.0 wt %. Accordingly, in some embodiments, the pharmaceutical composition is formulated for topical administration and comprises one or more antibiotic in an amount between about 0.1 and about 2.5 wt % of the total weight of the composition. In other embodiments, the pharmaceutical composition is formulated for topical administration and comprises one or more antibiotic in an amount between about 0.1 and about 1.0 wt % of the total weight of the composition. In still further embodiments, the pharmaceutical composition is formulated for topical administration and comprises one or more antibiotic in an amount of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, or about 5.0 wt % of the total weight of the composition. In yet other embodiments, the pharmaceutical composition is formulated for topical administration and comprises one or more antibiotic in an amount of about 0.51 wt % of the total weight of the composition. For the injectable pharmaceutical compositions, the amount of the one or more antibiotic may, for example, be between about 0.001 and about 1.0 wt % of the composition. In some embodiments, the pharmaceutical composition is formulated for injection and comprises one or antibiotic in an amount of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or about 1.0 wt % of the total weight of the composition.

The pharmaceutical compositions of the disclosure can also include one or more anesthetic compound typically used in the formulation of a pharmaceutical. Examples of anesthetic compounds for use in accordance with the disclosure include, but are not limited to, lidocaine, tetracaine, proparacaine, procaine, dyclonine, chloroprocaine, and any combination thereof. In some embodiments, disclosed herein is a pharmaceutical composition comprising an anesthetic compound, wherein the anesthetic compound is lidocaine. Those having ordinary skill in the art may choose other anesthetic compounds, if desired.

When present, the amount of the anesthetic compound(s) in the pharmaceutical compositions of the present disclosure may differ depending on whether the composition is intended for topical application or for intraocular injections. For topical compositions, the amount of the anesthetic compound may, for example, be between about 0.01 and about 7.0 wt %. In some embodiments, the pharmaceutical composition is formulated for topical application and the amount of the anesthetic compound may be about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, or about 7.0 wt % of the total weight of the composition. For the injectable compositions, the amount of the anesthetic compound may, likewise, be between about 0.01 and about 7.0 wt % of the composition. Accordingly, in some embodiments, the pharmaceutical composition is formulated for injection and comprises one or more anesthetic compound in an amount between about 0.1 and about 5.0 wt % of the total weight of the composition. In some additional embodiments, the pharmaceutical composition is formulated for injection and the amount of the anesthetic compound may be about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, or about 7.0 wt % of the total weight of the composition. In other embodiments, the pharmaceutical composition is formulated for injection and comprises one or more anesthetic compound in an amount between about 0.1 and about 2.5 wt % of the total weight of the composition. In yet other embodiments, the pharmaceutical composition is formulated for injection and comprises one or more anesthetic compound in an amount of about 0.98 wt % of the total weight of the composition.

The pharmaceutical compositions of the disclosure can also include one or more chelating agents, such as a metal chelator. Chelating agents suitable for use in the pharmaceutical compositions of the disclosure include those typically used in the formulation of a pharmaceutical. One non-limiting example of an acceptable chelating agent that may be used in combination with mydriatic compounds of the instant specification is ethylenediaminetetraacetic acid (EDTA), or pharmaceutically acceptable salts thereof, which is both a chelator and a stabilizer. Exemplary salt forms of EDTA suitable for use in the pharmaceutical compositions of the disclosure include, but are not limited to, EDTA disodium (i.e., edetate disodium), EDTA calcium disodium (i.e., sodium calcium edetate), and EDTA magnesium disodium, may be also used instead of, or in combination with, EDTA disodium. In some embodiments, disclosed herein is a pharmaceutical composition comprising a chelating agent, wherein the chelating agent is edetate disodium. When present, the amount of the chelating agent(s) may, for example, be between about 0.01 and about 2.0 wt %. Accordingly, in some embodiments, the pharmaceutical composition comprises one or more chelating agent in an amount between about 0.01 and about 0.7 wt % of the total weight of the composition. In some additional embodiments, the pharmaceutical composition comprises one or more chelating agent in an amount of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or about 2.0 wt % of the total weight of the composition. In other embodiments, the pharmaceutical composition comprises one or more chelating agent in an amount between about 0.05 and about 0.15 wt % of the total weight of the composition. In yet other embodiments, the pharmaceutical composition comprises one or more chelating agent in an amount of about 0.93 wt % of the total weight of the composition. In still other embodiments, the pharmaceutical composition comprises one or more chelating agent in an amount of about 0.98 wt % of the total weight of the composition.

The pharmaceutical compositions of the disclosure can also include one or more antioxidant typically used in the formulation of a pharmaceutical. Non-limiting examples of acceptable antioxidants that may be so used in the pharmaceutical compositions of the disclosure include ascorbic acid, vitamin E, glutathione and acetylcysteine. It must be kept in mind that these additional antioxidants typically provide only limited stability to mydriatic compounds, usually up to 60 days, when the pharmaceutical composition is kept refrigerated.

The pharmaceutical compositions of the disclosure can also comprise additional components, including excipients, adjuvants, diluents, modifiers, buffering agents, lubricants, demulcents, emulsifiers, and hypertonic agents. Accordingly, the pharmaceutical compositions of the disclosure may, by non-limiting example, include one or more of the following excipients: monosodium phosphate, disodium phosphate, monopotassium phosphate, dipotassium phosphate, phosphate, citric acid, boric acid, glycerin, hypromellose, polyethylene glycol 300, polyethylene glycol 400, carboxymethyl cellulose, hydroxyethyl cellulose, methylcellulose, dextran 70, polysorbate 80, propylene glycol, gelatin, polyvinyl alcohol, povidone, and sodium chloride. Accordingly, in some embodiments, disclosed herein is a pharmaceutical composition comprising boric acid. In some embodiments, disclosed herein is a pharmaceutical composition comprising powdered boric acid. In some embodiments, disclosed herein is a pharmaceutical composition comprising glycerin. In some embodiments, disclosed herein is a pharmaceutical composition comprising polysorbate 80. In some embodiments, disclosed herein is a pharmaceutical composition comprising sodium chloride. Those having ordinary skill in the art may choose other suitable components, if desired.

The pharmaceutically acceptable carrier may be any substance that serves as a vehicle for improving the efficiency of delivery and the effectiveness of the pharmaceutical composition. Preferably, the pharmaceutical compositions of the disclosure are aqueous formulations. Accordingly, in some embodiments, the pharmaceutically acceptable carrier is water (e.g., de-ionized sterile water, water sterile for injection).

The pharmaceutical compositions described in the present application, while being substantially free of both preservatives and sulfites, may contain preservatives or sulfites in trace or relatively low amounts. For instance, provided herein are pharmaceutical compositions suitable for intraocular injections that are 100% free of sulfites but may contain some limited quantity of other kinds of preservatives, if necessary. Also provided herein are pharmaceutical compositions suitable for topical administration that may contain some quantity of sulfites or preservatives, or both, if necessary.

Accordingly, in some embodiments, the pharmaceutical composition is substantially free of preservatives and are optionally free of sulfites.

In some embodiments, the pharmaceutical composition is free of preservatives. In other embodiments, the pharmaceutical composition is substantially free of preservatives. In yet other embodiments, the pharmaceutical composition comprises less than about 1.0 wt % preservatives by total weight of the composition; for example, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or less than 0.01 wt % of the total weight of the composition. In still other embodiments, the pharmaceutical composition comprises less than or equal to about 0.15 wt % preservatives by total weight of the composition.

In some embodiments, the pharmaceutical composition is free of sulfites. In other embodiments, the pharmaceutical composition is substantially free of sulfites. In yet other embodiments, the pharmaceutical composition comprises less than about 1.0 wt % sulfites by total weight of the composition; for example, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or less than 0.01 wt % of the total weight of the composition. In still other embodiments, the pharmaceutical composition comprises less than or equal to about 0.10 wt % sulfites by total weight of the composition.

Preservatives suitable for use in the pharmaceutical compositions of the disclosure include preservatives typically used in the formulation of a pharmaceutical. Examples of preservatives suitable for use in the pharmaceutical compositions disclosed herein include, but are not limited to, benzalkonium chloride, chlorobutanol, methyl paraben, sodium perborate, and thimerosal. In some embodiments, disclosed herein is a pharmaceutical composition comprising less than about 1 wt %, less than about 0.5 wt %, less than about 0.25 wt %, less than about 0.15 wt %, less than about 0.1 wt %, or less than about 0.05 wt % of a preservative, such as benzalkonium chloride. In some embodiments, disclosed herein is a pharmaceutical composition comprising less than or equal to about 0.15 wt %, less than or equal to about 0.1 wt %, or less than or equal to about 0.05 wt % of a preservative, such as benzalkonium chloride. In some embodiments, disclosed herein is a pharmaceutical composition comprising less than or equal to about 0.05 wt % of a preservative, such as benzalkonium chloride.

Sulfites suitable for use in the pharmaceutical compositions of the disclosure include sulfites typically used in the formulation of a pharmaceutical. Examples of sulfites suitable for use in the pharmaceutical compositions disclosed herein include, but are not limited to, sulfur dioxide, sodium sulfite, sodium bisulfite, potassium bisulfite, sodium metabisulfite, and potassium metabisulfite. In some embodiments, disclosed herein is a pharmaceutical composition comprising less than about 1 wt %, less than about 0.5 wt %, less than about 0.25 wt %, less than about 0.15 wt %, or less than about 0.1 wt % of a sulfite, such as sodium metabisulfite. In some embodiments, disclosed herein is a pharmaceutical composition comprising less than or equal to about 0.25 wt %, less than or equal to about 0.15 wt %, or less than or equal to about 0.1 wt % of a sulfite, such as sodium metabisulfite. In some embodiments, disclosed herein is a pharmaceutical composition comprising less than or equal to about 0.1 wt % of a sulfite, such as sodium metabisulfite.

In some embodiments, disclosed herein is a pharmaceutical composition comprising less than or equal to about 0.05 wt % of a preservative, such as benzalkonium chloride, and less than or equal to about 0.1 wt % of a sulfite, such as sodium metabisulfite. In some embodiments, disclosed herein is a pharmaceutical composition comprising less than or equal to about 0.15 wt %, collectively, of a preservative, such as benzalkonium chloride, and a sulfite, such as sodium metabisulfite.

In some embodiments, the disclosure provides a pharmaceutical composition comprising a therapeutically effective quantity of two mydriatic compounds, such as phenylephrine and tropicamide; a therapeutically effective quantity of at least one non-steroid anti-inflammatory drug (NSAID); optionally, a quantity of one or more gel forming compounds; and a pharmaceutically acceptable aqueous carrier. In further embodiments, the pharmaceutical composition is optionally free of preservatives and optionally free of sulfites, and the composition is formulated to be suitable for administration by an intraocular injection or for topical administration. In some embodiments, the pharmaceutical composition optionally further comprises one or more antibiotics, one or more anesthetic compounds, and one or more chelating agents.

In some embodiments, the disclosure provides a pharmaceutical composition comprising phenylephrine, or a pharmaceutically acceptable salt thereof; tropicamide, or a pharmaceutically acceptable salt thereof; an NSAID selected from the group consisting of ketorolac, diclofenac, or pharmaceutically acceptable salts thereof; optionally, one or more gelling agents selected from the group consisting of a poly(oxyethylene-co-oxypropylene) block copolymer, hydroxypropyl methylcellulose, and sodium alginate; and a pharmaceutically acceptable aqueous carrier; wherein the pharmaceutical composition is optionally free of preservatives and is optionally free of sulfites; and wherein the pharmaceutical composition is formulated to be suitable for administration by an intraocular injection or for topical administration.

In some embodiments, the pharmaceutical composition is formulated to be suitable for administration by topical administration and comprises one or more gelling agents selected from the group consisting of a poly(oxyethylene-co-oxypropylene) block copolymer, hydroxypropyl methylcellulose, and sodium alginate. In some embodiments, the pharmaceutical composition is formulated to be suitable for administration by intraocular injection and does not comprise any gel forming agents.

In some embodiments, the disclosure provides a pharmaceutical composition formulated to be suitable for administration by topical administration, wherein the pharmaceutical composition comprises phenylephrine, or a pharmaceutically acceptable salt thereof tropicamide, or a pharmaceutically acceptable salt thereof; ketorolac, or a pharmaceutically acceptable salt thereof; one or more of a poly(oxyethylene-co-oxypropylene) block copolymer, hydroxypropyl methylcellulose, and sodium alginate; and a pharmaceutically acceptable aqueous carrier; wherein the pharmaceutical composition is substantially free of preservatives and substantially free of sulfites. In some embodiments, the pharmaceutical composition further comprises one or more of moxifloxacin, or a pharmaceutically acceptable salt thereof; EDTA, or a pharmaceutically acceptable salt thereof; boric acid, glycerin; polysorbate 80; sodium metabisulfite; and benzalkonium chloride.

In some embodiments, the pharmaceutical composition formulated for topical administration comprises about 0.5 to about 5.0 wt %, collectively, of phenylephrine and tropicamide, or pharmaceutically acceptable salts thereof. In some embodiments, the pharmaceutical composition formulated for topical administration comprises about 0.5, 0.6, 0.7, 0.8, 0.9, 1.0, 1.5, 2.0, 2.5, 3.0, 3.5, 4.0, 4.5, or about 5.0 wt %, collectively, of phenylephrine and tropicamide, or pharmaceutically acceptable salts thereof. In some additional embodiments, the pharmaceutical composition comprises about 1.5 to about 4.5 wt %, collectively, of phenylephrine and tropicamide, or pharmaceutically acceptable salts thereof. In some embodiments, the pharmaceutical composition comprises about 3.3 wt %, collectively, of phenylephrine and tropicamide, or pharmaceutically acceptable salts thereof. In some embodiments, the pharmaceutical composition comprises 3.3 wt %, collectively, of phenylephrine and tropicamide, or pharmaceutically acceptable salts thereof.

In some embodiments, the pharmaceutical composition formulated for topical administration comprises about 1.3 to about 3.3 wt % phenylephrine, or a pharmaceutically acceptable salt thereof. In some embodiments, the pharmaceutical composition formulated for topical administration comprises about 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, or about 3.3 wt % phenylephrine, or a pharmaceutically acceptable salt thereof. In some additional embodiments, the pharmaceutical composition comprises about 1.8 to about 2.8 wt % phenylephrine, or a pharmaceutically acceptable salt thereof. In some embodiments, the pharmaceutical composition comprises about 2.3 wt % phenylephrine, or a pharmaceutically acceptable salt thereof. In some embodiments, the pharmaceutical composition comprises 2.3 wt % phenylephrine, or a pharmaceutically acceptable salt thereof.

In some embodiments, the pharmaceutical composition formulated for topical administration comprises about 0.1 to about 2.0 wt % tropicamide. In some embodiments, the pharmaceutical composition formulated for topical administration comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or about 2.0 wt % tropicamide. In some additional embodiments, the pharmaceutical composition comprises about 0.4 to about 1.4 wt % tropicamide. In some embodiments, the pharmaceutical composition comprises about 0.9 wt % tropicamide. In some embodiments, the pharmaceutical composition comprises 0.9 wt % tropicamide.

In some embodiments, the wt % ratio of phenylephrine, or the pharmaceutically acceptable salt thereof, to tropicamide is about 1:1, about 1.5:1, about 2:1, about 2.5:1, about 3:1, about 3.5:1, or about 4:1. In some embodiments, the wt % ratio of phenylephrine, or the pharmaceutically acceptable salt thereof, to tropicamide is from about 2:1 to about 3:1. In some embodiments, the wt % ratio of phenylephrine, or the pharmaceutically acceptable salt thereof, to tropicamide is about 2.1:1, about 2.2:1, about 2.3:1, about 2.4:1, about 2.5:1, about 2.6:1, about 2.7:1, about 2.8:1, or about 2.9:1. In some embodiments, the wt % ratio of phenylephrine, or the pharmaceutically acceptable salt thereof, to tropicamide is about 2.5:1.

In some embodiments, the pharmaceutical composition formulated for topical administration comprises about 0.1 to about 1.5 wt % ketorolac, or a pharmaceutically acceptable salt thereof. In some embodiments, the pharmaceutical composition formulated for topical administration comprises about 0.1, 0.2, 0.3, 0.4, 0.5, 0.6, 0.7, 0.8, 0.9, 1.0, 1.1, 1.2, 1.3, 1.4, or about 1.5 wt % ketorolac. In some additional embodiments, the pharmaceutical composition comprises about 0.1 to about 0.8 wt % ketorolac, or a pharmaceutically acceptable salt thereof. In some embodiments, the pharmaceutical composition comprises about 0.37 wt % ketorolac, or a pharmaceutically acceptable salt thereof. In some embodiments, the pharmaceutical composition comprises 0.37 wt % ketorolac, or a pharmaceutically acceptable salt thereof.

In some embodiments, the pharmaceutical composition formulated for topical administration comprises about 0.01 to about 1.5 wt % of a poly(oxyethylene-co-oxypropylene) block copolymer. In some embodiments, the pharmaceutical composition formulated for topical administration comprises about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, or about 1.5 wt % of a poly(oxyethylene-co-oxypropylene) block copolymer. In some additional embodiments, the pharmaceutical composition comprises about 0.1 to about 0.4 wt % of a poly(oxyethylene-co-oxypropylene) block copolymer. In some embodiments, the pharmaceutical composition comprises about 0.19 wt % of a poly(oxyethylene-co-oxypropylene) block copolymer. In some embodiments, the pharmaceutical composition comprises 0.19 wt % of a poly(oxyethylene-co-oxypropylene) block copolymer.

In some embodiments, the pharmaceutical composition formulated for topical administration comprises about 0.01 to about 1.5 wt % hydroxypropyl methylcellulose. In some embodiments, the pharmaceutical composition formulated for topical administration comprises about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, or about 1.5 wr/o hydroxypropyl methylcellulose. In some additional embodiments, the pharmaceutical composition comprises about 0.1 to about 0.4 wt % hydroxypropyl methylcellulose. In some embodiments, the pharmaceutical composition comprises about 0.23 wt % hydroxypropyl methylcellulose. In some embodiments, the pharmaceutical composition comprises 0.23 wt % hydroxypropyl methylcellulose.

In some embodiments, the pharmaceutical composition formulated for topical administration comprises about 0.01 to about 1.5 wt % sodium alginate. In some embodiments, the pharmaceutical composition formulated for topical administration comprises about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, or about 1.5 wt % sodium alginate. In some additional embodiments, the pharmaceutical composition comprises about 0.1 to about 0.4 wt % sodium alginate. In some embodiments, the pharmaceutical composition comprises about 0.23 wt % sodium alginate. In some embodiments, the pharmaceutical composition comprises 0.23 wt % sodium alginate.

In some embodiments, the pharmaceutical composition formulated for topical administration comprises about 0.1 to about 2.5 wt % moxifloxacin, or a pharmaceutically acceptable salt thereof. In some embodiments, the pharmaceutical composition formulated for topical administration comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or about 2.5 wt % moxifloxacin, or a pharmaceutically acceptable salt thereof. In some additional embodiments, the pharmaceutical composition comprises about 0.1 to about 1.0 wt % moxifloxacin, or a pharmaceutically acceptable salt thereof. In some embodiments, the pharmaceutical composition comprises about 0.51 wt % moxifloxacin, or a pharmaceutically acceptable salt thereof. In some embodiments, the pharmaceutical composition comprises 0.51 wt % moxifloxacin, or a pharmaceutically acceptable salt thereof.

In some embodiments, the pharmaceutical composition formulated for topical administration comprises about 0.01 to about 0.7 wt % EDTA, or a pharmaceutically acceptable salt thereof. In some embodiments, the pharmaceutical composition formulated for topical administration comprises about 0.01, 0.02, 0.03, 0.04, 0.05, 0.06, 0.07, 0.08, 0.09, 0.1, 0.2, 0.3, 0.4, 0.5, 0.6, or about 0.7 wt % EDTA. In some additional embodiments, the pharmaceutical composition comprises about 0.05 to about 0.15 wt % EDTA, or a pharmaceutically acceptable salt thereof. In some embodiments, the pharmaceutical composition comprises about 0.09 wt % EDTA, or a pharmaceutically acceptable salt thereof. In some embodiments, the pharmaceutical composition comprises 0.09 wt % EDTA, or a pharmaceutically acceptable salt thereof.

In some embodiments, the pharmaceutical composition formulated for topical administration comprises about 0.1 to about 1.5 wt % boric acid. In some embodiments, the pharmaceutical composition formulated for topical administration comprises about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, or about 1.5 wt′Yo boric acid. In some additional embodiments, the pharmaceutical composition comprises about 0.1 to about 0.8 wt % boric acid. In some embodiments, the pharmaceutical composition comprises about 0.3 wt % boric acid. In some embodiments, the pharmaceutical composition comprises 0.3 wt % boric acid.

In some embodiments, the pharmaceutical composition formulated for topical administration comprises about 0.1 to about 5.0 wt % glycerin. In some embodiments, the pharmaceutical composition formulated for topical administration comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0. 3.5. 4.0. 4.5, or about 5.0 wt % glycerin. In some additional embodiments, the pharmaceutical composition comprises about 0.25 to about 1.75 wt % glycerin. In some embodiments, the pharmaceutical composition comprises about 1.2 wt % glycerin. In some embodiments, the pharmaceutical composition comprises 1.2 wt % glycerin.

In some embodiments, the pharmaceutical composition formulated for topical administration comprises about 0.01 to about 1.5 wt % polysorbate 80. In some embodiments, the pharmaceutical composition formulated for topical administration comprises about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, or about 1.5 wt % polysorbate 80. In some additional embodiments, the pharmaceutical composition comprises about 0.05 to about 0.5 wt % polysorbate 80. In some embodiments, the pharmaceutical composition comprises about 0.2 wt % polysorbate 80. In some embodiments, the pharmaceutical composition comprises 0.2 wt % polysorbate 80.

In some embodiments, the pharmaceutical composition formulated for topical administration comprises less than or equal to about 0.15 wt % benzalkonium chloride; for example, the pharmaceutical composition formulated for topical administration may comprise about 0.15, 0.14, 0.13, 0.12, 0.11, 0.10, 0.09, 0.08, 0.07, 0.06, 0.05, 0.04, 0.03, 0.02, 0.01, or less than 0.01 wt % benzalkonium chloride. In some embodiments, the pharmaceutical composition comprises less than or equal to about 0.1 wt % benzalkonium chloride. In some embodiments, the pharmaceutical composition comprises less than or equal to about 0.05 wt % benzalkonium chloride.

In some embodiments, the pharmaceutical composition formulated for topical administration comprises less than or equal to about 0.25 wt % sodium metabisulfite; for example, the pharmaceutical composition formulated for topical administration may comprise about 0.25, 0.24, 0.23, 0.22, 0.21, 0.20, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or less than 0.01 wt % sodium metabisulfite. In some embodiments, the pharmaceutical composition comprises less than or equal to about 0.15 wt % sodium metabisulfite. In some embodiments, the pharmaceutical composition comprises less than or equal to about 0.1 wt % sodium metabisulfite.

In some embodiments, the pharmaceutical composition formulated for topical administration comprises less than or equal to about 0.25 wt %, collectively, of benzalkonium chloride and sodium metabisulfite; for example, the pharmaceutical composition formulated for topical administration may comprise about 0.25, 0.24, 0.23, 0.22, 0.21, 0.20, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or less than 0.01 wt %, collectively, of benzalkonium chloride and sodium metabisulfite. In some embodiments, the pharmaceutical composition comprises less than or equal to about 0.20 wt %, collectively, of benzalkonium chloride and sodium metabisulfite. In some embodiments, the pharmaceutical composition comprises less than or equal to about 0.15 wt %, collectively, of benzalkonium chloride and sodium metabisulfite. In some embodiments, the pharmaceutical composition comprises less than or equal to about 0.05 wt % benzalkonium chloride and less than or equal to about 0.1 wt % sodium metabisulfite.

In some embodiments, the pharmaceutical composition formulated for topical administration comprises about 1.8 to about 2.8 wt % phenylephrine, or a pharmaceutically acceptable salt thereof; about 0.4 to about 1.4 wt % tropicamide; about 0.1 to about 0.8 wt % ketorolac, or a pharmaceutically acceptable salt thereof; about 0.1 to about 0.4 wt % of a poly(oxyethylene-co-oxypropylene) block copolymer; about 0.1 to about 0.4 wt % hydroxypropyl methylcellulose; about 0.1 to about 0.4 wt % sodium alginate; and a pharmaceutically acceptable aqueous carrier; wherein the pharmaceutical composition is substantially free of preservatives and substantially free of sulfites. In some embodiments, the pharmaceutical composition further comprises about 0.1 to about 1.0 wt % moxifloxacin, or a pharmaceutically acceptable salt thereof; about 0.05 to about 0.15 wt % EDTA, or a pharmaceutically acceptable salt thereof; about 0.1 to about 0.8 wt % boric acid; about 0.25 to about 1.75 wt % glycerin; about 0.05 to about 0.5 wt % polysorbate 80; less than or equal to about 0.1 wt % benzalkonium chloride; and less than or equal to about 0.15 wt % sodium metabisulfite.

In some embodiments, the pharmaceutical composition formulated for topical administration comprises about 2.3 wt % phenylephrine, or a pharmaceutically acceptable salt thereof; about 0.9 wt % tropicamide; about 0.37 wt % ketorolac, or a pharmaceutically acceptable salt thereof; about 0.4 wt % of a poly(oxyethylene-co-oxypropylene) block copolymer; about 0.23 wt % hydroxypropyl methylcellulose; about 0.23 wt % sodium alginate; and a pharmaceutically acceptable aqueous carrier; wherein the pharmaceutical composition is substantially free of preservatives and substantially free of sulfites. In some embodiments, the pharmaceutical composition further comprises about 0.51 wt % moxifloxacin, or a pharmaceutically acceptable salt thereof; about 0.09 wt % EDTA, or a pharmaceutically acceptable salt thereof; about 0.3 wt % boric acid; about 1.2 wt % glycerin; about 0.2 wt % polysorbate 80; less than or equal to about 0.05 wt % benzalkonium chloride; and less than or equal to about 0.1 wt % sodium metabisulfite.

In some embodiments, the pharmaceutical composition formulated for topical administration comprises 2.3 wt % phenylephrine, or a pharmaceutically acceptable salt thereof; 0.9 wt % tropicamide; 0.37 wt % ketorolac, or a pharmaceutically acceptable salt thereof; 0.4 wt % of a poly(oxyethylene-co-oxypropylene) block copolymer; 0.23 wt % hydroxypropyl methylcellulose; 0.23 wt % sodium alginate; and a pharmaceutically acceptable aqueous carrier; wherein the pharmaceutical composition is substantially free of preservatives and substantially free of sulfites. In some embodiments, the pharmaceutical composition further comprises 0.51 wt % moxifloxacin, or a pharmaceutically acceptable salt thereof; 0.09 wt % EDTA, or a pharmaceutically acceptable salt thereof; 0.3 wt % boric acid; 1.2 wt % glycerin; 0.2 wt % polysorbate 80; less than or equal to 0.05 wt % benzalkonium chloride; and less than or equal to 0.1 wt % sodium metabisulfite.

In some embodiments, the pharmaceutical composition formulated for topical administration comprises about 1.0 to about 5.0 g phenylephrine, or a pharmaceutically acceptable salt thereof; about 0.5 to about 2.0 g tropicamide; about 0.1 to about 1.0 g ketorolac, or a pharmaceutically acceptable salt thereof; about 0.1 to about 0.5 g of a poly(oxyethylene-co-oxypropylene) block copolymer; about 0.1 to about 0.5 g hydroxypropyl methylcellulose; about 0.1 to about 0.5 g sodium alginate; and about 90 to about 110 mL water; wherein the pharmaceutical composition is substantially free of preservatives and substantially free of sulfites. In some embodiments, the pharmaceutical composition further comprises about 0.1 to about 1.0 g moxifloxacin, or a pharmaceutically acceptable salt thereof; about 0.05 to about 0.2 g EDTA, or a pharmaceutically acceptable salt thereof; about 0.1 to about 0.7 g boric acid; about 0.5 to about 2.0 mL glycerin; about 0.1 to about 0.5 mL polysorbate 80; about 0.1 to about 0.9 mL of a 1% aqueous solution of benzalkonium chloride (i.e., about 0.01 to about 0.09 g benzalkonium chloride); and about 0.05 to about 0.15 g sodium metabisulfite.

In some embodiments, the pharmaceutical composition formulated for topical administration comprises about 2.5 g phenylephrine, or a pharmaceutically acceptable salt thereof; about 1.0 g tropicamide; about 0.4 g ketorolac, or a pharmaceutically acceptable salt thereof; about 0.2 g of a poly(oxyethylene-co-oxypropylene) block copolymer; about 0.25 g hydroxypropyl methylcellulose; about 0.25 g sodium alginate; and about 100 mL water; wherein the pharmaceutical composition is substantially free of preservatives and substantially free of sulfites. In some embodiments, the pharmaceutical composition further comprises about 0.545 g moxifloxacin, or a pharmaceutically acceptable salt thereof; about 0.1 g EDTA, or a pharmaceutically acceptable salt thereof; about 0.352 g boric acid; about 1.0 mL glycerin; about 0.2 mL polysorbate 80; about 0.5 mL of a 1% aqueous solution of benzalkonium chloride (i.e., about 0.05 g benzalkonium chloride); and about 0.1 g sodium metabisulfite.

In some embodiments, the pharmaceutical composition formulated for topical administration comprises 2.5 g phenylephrine, or a pharmaceutically acceptable salt thereof; 1.0 g tropicamide; 0.4 g ketorolac, or a pharmaceutically acceptable salt thereof; 0.2 g of a poly(oxyethylene-co-oxypropylene) block copolymer; 0.25 g hydroxypropyl methylcellulose; 0.25 g sodium alginate; and 100 mL water; wherein the pharmaceutical composition is substantially free of preservatives and substantially free of sulfites. In some embodiments, the pharmaceutical composition further comprises 0.545 g moxifloxacin, or a pharmaceutically acceptable salt thereof; 0.1 g EDTA, or a pharmaceutically acceptable salt thereof; 0.352 g boric acid; 1.0 mL glycerin; 0.2 mL polysorbate 80; 0.5 mL of a 1% aqueous solution of benzalkonium chloride (i.e., 0.05 g benzalkonium chloride); and 0.1 g sodium metabisulfite.

In some embodiments, the phenylephrine is phenylephrine hydrochloride. In some embodiments, the ketorolac is ketorolac tromethamine. In some embodiments, the poly(oxyethylene-co-oxypropylene) block copolymer is POLOXAMER® 407. In some embodiments, hydroxypropyl methylcellulose is METHOCEL® E4M. In some embodiments, the sodium alginate is powdered sodium alginate. In some embodiments, the moxifloxacin is moxifloxacin hydrochloride monohydrate. In some embodiments, the EDTA is edetate disodium. In some embodiments, the boric acid is powdered boric acid. In some embodiments, the sodium metabisulfite is granulated sodium metabisulfite.

In some embodiments, the disclosure provides a pharmaceutical composition formulated to be suitable for administration by intraocular injection, wherein the pharmaceutical composition comprises phenylephrine, or a pharmaceutically acceptable salt thereof; tropicamide, or a pharmaceutically acceptable salt thereof; diclofenac, or a pharmaceutically acceptable salt thereof; and a pharmaceutically acceptable aqueous carrier; wherein the pharmaceutical composition is substantially free of preservatives and substantially free of sulfites. In some embodiments, the pharmaceutical composition further comprises one or more of lidocaine, or a pharmaceutically acceptable salt thereof; EDTA, or a pharmaceutically acceptable salt thereof; boric acid, polysorbate 80; and sodium chloride.

In some embodiments, the pharmaceutical composition formulated for intraocular injection comprises about 0.1 to about 1.0 wt %, collectively, of phenylephrine and tropicamide, or pharmaceutically acceptable salts thereof. In some embodiments, the pharmaceutical formulation formulated for intraocular injection comprises about 0.1, 0.2, 0.3, 0.4, 0.5, 0.6, 0.7, 0.8, 0.9, or about 1.0 wt %, collectively, of phenylephrine and tropicamide, or pharmaceutically acceptable salts thereof. In some additional embodiments, the pharmaceutical composition comprises about 0.1 to about 0.5 wt %, collectively, of phenylephrine and tropicamide, or pharmaceutically acceptable salts thereof. In some embodiments, the pharmaceutical composition comprises about 0.3 wt %, collectively, of phenylephrine and tropicamide, or pharmaceutically acceptable salts thereof. In some embodiments, the pharmaceutical composition comprises 0.3 wt %, collectively, of phenylephrine and tropicamide, or pharmaceutically acceptable salts thereof.

In some embodiments, the pharmaceutical composition formulated for intraocular injection comprises about 0.1 to about 1.0 wt % phenylephrine, or a pharmaceutically acceptable salt thereof. In some embodiments, the pharmaceutical formulation formulated for intraocular injection comprises about 0.1, 0.2, 0.3, 0.4, 0.5, 0.6, 0.7, 0.8, 0.9, or about 1.0 wt % phenylephrine, or a pharmaceutically acceptable salt thereof. In some additional embodiments, the pharmaceutical composition comprises about 0.1 to about 0.5 wt % phenylephrine, or a pharmaceutically acceptable salt thereof. In some embodiments, the pharmaceutical composition comprises about 0.29 wt % phenylephrine, or a pharmaceutically acceptable salt thereof. In some embodiments, the pharmaceutical composition comprises 0.29 wt % phenylephrine, or a pharmaceutically acceptable salt thereof.

In some embodiments, the pharmaceutical composition formulated for intraocular injection comprises about 0.005 to about 0.5 wt % tropicamide. In some embodiments, the pharmaceutical composition formulated for intraocular injection comprises about 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, or about 0.5 wt % tropicamide. In some additional embodiments, the pharmaceutical composition comprises about 0.01 to about 0.03 wt % tropicamide. In some embodiments, the pharmaceutical composition comprises about 0.02 wt % tropicamide. In some embodiments, the pharmaceutical composition comprises 0.02 wt % tropicamide.

In some embodiments, the wt % ratio of phenylephrine, or the pharmaceutically acceptable salt thereof, to tropicamide is about 1:1, about 5:1 about 10:1, about 15:1, about 20:1, about 25:1, or about 30:1. In some embodiments, the wt % ratio of phenylephrine, or the pharmaceutically acceptable salt thereof, to tropicamide is from about 10:1 to about 20:1. In some embodiments, the wt % ratio of phenylephrine, or the pharmaceutically acceptable salt thereof, to tropicamide is about 11:1, about 12:1, about 13:1, about 14:1, about 15:1, about 16:1, about 17:1, about 18:1, or about 19:1. In some embodiments, the wt % ratio of phenylephrine, or the pharmaceutically acceptable salt thereof, to tropicamide is about 15:1.

In some embodiments, the pharmaceutical composition formulated for intraocular injection comprises about 0.005 to about 0.5 wt % diclofenac, or a pharmaceutically acceptable salt thereof. In some embodiments, the pharmaceutical composition formulated for intraocular injection comprises about 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, or about 0.5 wt % diclofenac, or a pharmaceutically acceptable salt thereof. In some additional embodiments, the pharmaceutical composition comprises about 0.007 to about 0.013 wt % diclofenac, or a pharmaceutically acceptable salt thereof. In some embodiments, the pharmaceutical composition comprises about 0.01 wt % diclofenac, or a pharmaceutically acceptable salt thereof. In some embodiments, the pharmaceutical composition comprises 0.01 wt % diclofenac, or a pharmaceutically acceptable salt thereof.

In some embodiments, the pharmaceutical composition formulated for intraocular injection comprises about 0.1 to about 5.0 wt % lidocaine. In some embodiments, the pharmaceutical composition formulated for intraocular injection comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, or about 5.0 wt % lidocaine. In some additional embodiments, the pharmaceutical composition comprises about 0.1 to about 2.5 wt % lidocaine. In some embodiments, the pharmaceutical composition comprises about 0.98 wt % lidocaine. In some embodiments, the pharmaceutical composition comprises 0.98 wt % lidocaine.

In some embodiments, the pharmaceutical composition formulated for intraocular injection comprises about 0.01 to about 0.7 wt % EDTA, or a pharmaceutically acceptable salt thereof. In some embodiments, the pharmaceutical composition formulated for intraocular injection comprises about 0.01, 0.02, 0.03, 0.04, 0.05, 0.06, 0.07, 0.08, 0.09, 0.1, 0.2, 0.3, 0.4, 0.5, 0.6, or about 0.7 wt % EDTA, or a pharmaceutically acceptable salt thereof. In some additional embodiments, the pharmaceutical composition comprises about 0.05 to about 0.15 wt % EDTA, or a pharmaceutically acceptable salt thereof. In some embodiments, the pharmaceutical composition comprises about 0.1 wt % EDTA, or a pharmaceutically acceptable salt thereof. In some embodiments, the pharmaceutical composition comprises 0.1 wt % EDTA, or a pharmaceutically acceptable salt thereof.

In some embodiments, the pharmaceutical composition formulated for intraocular injection comprises about 0.1 to about 1.5 wt % boric acid. In some embodiments, the pharmaceutical composition formulated for intraocular injection comprises about 0.1, 0.2, 0.3, 0.4, 0.5, 0.6, 0.7, 0.8, 0.9, 1.0, 1.1, 1.2, 1.3, 1.4, or about 1.5 wt % boric acid. In some additional embodiments, the pharmaceutical composition comprises about 0.1 to about 0.8 wt % boric acid. In some embodiments, the pharmaceutical composition comprises about 0.3 wt % boric acid. In some embodiments, the pharmaceutical composition comprises 0.3 wt % boric acid.

In some embodiments, the pharmaceutical composition formulated for intraocular injection comprises about 0.01 to about 1.5 wt % polysorbate 80. In some embodiments, the pharmaceutical composition formulated for intraocular injection comprises about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, or about 1.5 wt % polysorbate 80. In some additional embodiments, the pharmaceutical composition comprises about 0.05 to about 0.5 wt % polysorbate 80. In some embodiments, the pharmaceutical composition comprises about 0.1 wt % polysorbate 80. In some embodiments, the pharmaceutical composition comprises 0.1 wt % polysorbate 80.

In some embodiments, the pharmaceutical composition formulated for intraocular injection comprises about 0.1 to about 1.5 wt % sodium chloride. In some embodiments, the pharmaceutical composition formulated for intraocular injection comprises about 0.1, 0.2, 0.3, 0.4, 0.5, 0.6, 0.7, 0.8, 0.9, 1.0, 1.1, 1.2, 1.3, 1.4, or about 1.5 wt % sodium chloride. In some additional embodiments, the pharmaceutical composition comprises about 0.1 to about 0.8 wt % sodium chloride. In some embodiments, the pharmaceutical composition comprises about 0.38 wt % sodium chloride. In some embodiments, the pharmaceutical composition comprises 0.38 wt % sodium chloride.

In some embodiments, the pharmaceutical composition formulated for intraocular injection is preservative free. In some embodiments, the pharmaceutical composition is sulfite free.

In some embodiments, the pharmaceutical composition formulated for intraocular injection comprises about 0.1 to about 0.5 wt % phenylephrine, or a pharmaceutically acceptable salt thereof; about 0.01 to about 0.03 wt % tropicamide; about 0.007 to about 0.013 wt % diclofenac, or a pharmaceutically acceptable salt thereof; and a pharmaceutically acceptable aqueous carrier; wherein the pharmaceutical composition is substantially free of preservatives and substantially free of sulfites. In some embodiments, the pharmaceutical composition further comprises about 0.1 to about 2.5 wt % lidocaine; about 0.05 to about 0.15 wt % EDTA, or a pharmaceutically acceptable salt thereof; about 0.1 to about 0.8 wt % boric acid; about 0.05 to about 0.5 wt % polysorbate 80; and about 0.1 to about 0.8 wt % sodium chloride.

In some embodiments, the pharmaceutical composition formulated for intraocular injection comprises about 0.29 wt % phenylephrine, or a pharmaceutically acceptable salt thereof; about 0.02 wt % tropicamide; about 0.01 wt % diclofenac, or a pharmaceutically acceptable salt thereof; and a pharmaceutically acceptable aqueous carrier; wherein the pharmaceutical composition is preservatives free and sulfite free. In some embodiments, the pharmaceutical composition further comprises about 0.98 wt % lidocaine; about 0.1 wt % EDTA, or a pharmaceutically acceptable salt thereof; about 0.3 wt % boric acid; about 0.1 wt % polysorbate 80; and about 0.38 wt % sodium chloride.

In some embodiments, the pharmaceutical composition formulated for intraocular injection comprises 0.29 wt % phenylephrine, or a pharmaceutically acceptable salt thereof; 0.02 wt % tropicamide; 0.01 wt % diclofenac, or a pharmaceutically acceptable salt thereof; and a pharmaceutically acceptable aqueous carrier; wherein the pharmaceutical composition is preservatives free and sulfite free. In some embodiments, the pharmaceutical composition further comprises 0.98 wt % lidocaine; 0.1 wt % EDTA, or a pharmaceutically acceptable salt thereof; 0.3 wt % boric acid; 0.1 wt % polysorbate 80; and 0.38 wt % sodium chloride.

In some embodiments, the pharmaceutical composition formulated for intraocular injection comprises about 0.1 to about 0.6 g phenylephrine, or a pharmaceutically acceptable salt thereof; about 0.01 to about 0.05 g tropicamide; about 0.005 to about 0.02 g diclofenac, or a pharmaceutically acceptable salt thereof; and about 90 to about 110 mL water; wherein the pharmaceutical composition is substantially free of preservatives and substantially free of sulfites. In some embodiments, the pharmaceutical composition further comprises about 0.5 to about 2.0 g lidocaine; about 0.05 to about 0.2 g EDTA, or a pharmaceutically acceptable salt thereof; about 0.1 to about 0.7 g boric acid; about 0.05 to about 0.2 mL polysorbate 80; and about 0.1 to about 0.7 g sodium chloride.

In some embodiments, the pharmaceutical composition formulated for intraocular injection comprises about 0.3 g phenylephrine, or a pharmaceutically acceptable salt thereof; about 0.02 g tropicamide; about 0.01 g diclofenac, or a pharmaceutically acceptable salt thereof; and about 100 mL water; wherein the pharmaceutical composition is preservative free and sulfite free. In some embodiments, the pharmaceutical composition further comprises about 1.0 g lidocaine; about 0.1 g EDTA, or a pharmaceutically acceptable salt thereof; about 0.352 g boric acid; about 0.1 mL polysorbate 80; and about 0.386 g sodium chloride.

In some embodiments, the pharmaceutical composition formulated for intraocular injection comprises 0.3 g phenylephrine, or a pharmaceutically acceptable salt thereof; 0.02 g tropicamide; 0.01 g diclofenac, or a pharmaceutically acceptable salt thereof; and 100 mL water; wherein the pharmaceutical composition is preservative free and sulfite free. In some embodiments, the pharmaceutical composition further comprises 1.0 g lidocaine; 0.1 g EDTA, or a pharmaceutically acceptable salt thereof; 0.352 g boric acid; 0.1 mL polysorbate 80; and 0.386 g sodium chloride.

In some embodiments, the phenylephrine is phenylephrine hydrochloride. In some embodiments, the diclofenac is diclofenac sodium. In some embodiments, the EDTA is edetate disodium. In some embodiments, the boric acid is powdered boric acid. In some embodiments, the sodium chloride is granulated sodium chloride.

The pharmaceutical compositions disclosed herein are intended either for topical ophthalmic administration or for administration by an intraocular injection. Typically, topical drops may require a substantial time to be effective, up to 45 minutes or even longer. Without committing to any particular scientific or medical theory, for the topical compositions of the present disclosure a gel forms on the surface of the eye, creating enhanced corneal penetration and a quicker onset of mydriasis. Additionally, it prolongs the effect for the same reason. The use of a quantity of a gel forming compound is required for pharmaceutical compositions intended for topical applications.

For the pharmaceutical compositions intended for intracameral injection an immediate mydriasis occurs (within about 30 seconds after the injection) leading to a prolonged mydriasis because it is injected directly into the site of action and does not require penetration through ocular membranes or structures. No gel forming compound is to be used for injectable compositions.

A variety of suitable methods are envisioned that one having ordinary skill in the art can employ to prepare preservative-free and sulfite-free pharmaceutical compositions described above. According to one non-limiting embodiment, a one batch method may be used, i.e., when all the components are mixed in the same container.

It will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, gender, diet, and the severity of the particular ophthalmological condition being treated.

The pharmaceutical compositions disclosed herein, which are substantially free of preservatives and sulfites or free of preservatives and sulfites, are shelf stable under standard storage conditions employed in the industry. The pharmaceutical compositions of the disclosure may, for example, be stored at room temperature for at least 5 months, at least 6 months, at least 7 months, at least 8 months, at least 9 months, at least 12 months, at least 18 months, or at least 24 months without significant degradation of any of the active ingredients (e.g., mydriatic compounds, NSAIDs, antibiotics, anesthetics, etc.) comprised therein. In some embodiments, the pharmaceutical compositions of the disclosure can be stored at room temperature for at least 6 months without significant degradation of any of the active ingredients comprised therein. Accordingly, the pharmaceutical compositions of the disclosure may, for example, be stored at room temperature for a length of time disclosed herein without exhibiting more than 10%, more than 9%, more than 8%, more than 7%, more than 6%, more than 5%, more than 4%, more than 3%, more than 2%, or more than 1% reduction of any active ingredient, by weight, as a result of degradation.

In additional embodiments, pharmaceutical kits are provided. The kit includes a sealed container approved for the storage of pharmaceutical compositions, the container containing one of the above-described pharmaceutical compositions. An instruction for the use of the composition and the information about the composition are to be included in the kit.

### Exemplary Embodiments

Embodiment 1: A pharmaceutical composition, comprising:
(a) a therapeutically effective quantity of two mydriatic compounds;
(b) a therapeutically effective quantity of at least one non-steroid anti-inflammatory drug;
(c) optionally, a quantity of a one or more gel forming compounds;
(d) a pharmaceutically acceptable aqueous carrier,
wherein the pharmaceutical composition is optionally free of preservatives and is optionally free of sulfites, with the further proviso that the pharmaceutical composition is formulated to be suitable for administration by an intraocular injection or for topical administration.

Embodiment 2: The pharmaceutical composition of embodiment 1, wherein the two mydriatic compounds are selected from the group consisting of phenylephrine, tropicamide, brimonidine, cyclopentolate, cyclopentolate hydrochloride, atropine, homatropine, scopolamine; and pharmaceutically acceptable salts thereof.

Embodiment 3: The pharmaceutical composition of embodiment 1 or embodiment 2, wherein the two mydriatic compounds are phenylephrine and tropicamide, or pharmaceutically acceptable salts thereof.

Embodiment 4: The pharmaceutical composition of any of embodiments 1-3, wherein the non-steroid anti-inflammatory drug is selected from the group consisting of ketorolac, bromfenac, etodolac, sulindac, diclofenac, aceclofenac, nepafenac, tolmetin, indomethacin, nabumetone, ketoprofen, dexketoprofen, ibuprofen, flurbiprofen, dexibuprofen, fenoprofen, loxoprofen, oxaprozin, naproxen, aspirin, salicylic acid, diflunisal, salsalate, mefenamic acid, meclofenamic acid, flufenamic acid, tolfenamic acid, meloxicam, piroxicam, ternoxicam, droxicam, lornoxicam, isoxicam, celecoxib, rofecoxib, valdecoxib, parecoxib, lumiracoxib, etoricoxib, firocoxib, nimesulide, clonixin, licofelone, and pharmaceutically acceptable salts thereof.

Embodiment 5: The pharmaceutical composition of any of embodiments 1-4, wherein the non-steroid anti-inflammatory drug is ketorolac, or a pharmaceutically acceptable salt thereof.

Embodiment 6: The pharmaceutical composition of any of embodiments 1-4, wherein the non-steroid anti-inflammatory drug is diclofenac, or a pharmaceutically acceptable salt thereof.

Embodiment 7: The pharmaceutical composition of any of embodiments 1-6, wherein the one or more gel forming compounds is selected from the group consisting of alginic acid, sodium alginate, potassium alginate, calcium alginate, agar-agar, pectin, guar gum, xanthan gum, gelatin, poly(oxyethylene-co-oxypropylene) block copolymers, poly(N-isopropylacrylamide), poly(N-isopropylacrylamide-co-acrylic acid), poly(vinyl pyrrolidone), poly(-vinylpyridine-co-ethylacrylate) block copolymers, poly(N-isopropylacrylamide-co-butyl methacrylate-co-ethylene glycol) block copolymers, carboxymethyl cellulose hydroxyethyl cellulose, methyl cellulose, hydroxypropyl cellulose, hydroxypropyl methylcellulose, polyoxyethylene sorbitan monolaurates, polyoxyethylene sorbitan monopalmitates, polyoxyethylene sorbitan monostearates, and polyoxyethylene sorbitan monooleates.

Embodiment 8: The pharmaceutical composition of any one of embodiments 1-7, wherein the one or more gel forming compounds comprises sodium alginate.

Embodiment 9: The pharmaceutical composition of any one of embodiments 1-8, wherein the one or more gel forming compounds comprises a quantity of a poly(oxyethylene-co-oxypropylene) block copolymer.

Embodiment 10: The pharmaceutical composition of any one of embodiments 1-9, wherein the one or more gel forming compounds comprises hydroxypropyl methylcellulose.

Embodiment 11: The pharmaceutical composition of any one of embodiments 1-10, further comprising a therapeutically effective quantity of at least one anesthetic compound selected from the group consisting of lidocaine, tetracaine, proparacaine, procaine, dyclonine, chloroprocaine, and pharmaceutically acceptable salts thereof.

Embodiment 12: The pharmaceutical composition of embodiment 11, wherein the anesthetic compound is lidocaine, or a pharmaceutically acceptable salt thereof.

Embodiment 13: The pharmaceutical composition of any one of embodiments 1-12, further comprising a therapeutically effective quantity of at least one antibiotic selected from the group consisting of moxifloxacin, gatifloxacin, teicoplanin, telavancin, decaplanin, ramoplanin ciprofloxacin, besifloxacin, levofloxacin, gentamicin, tobramycin and amikacin, and pharmaceutically acceptable salts thereof.

Embodiment 14: The pharmaceutical composition of embodiment 13, wherein the antibiotic is moxifloxacin, or a pharmaceutically acceptable salt thereof.

Embodiment 15: The pharmaceutical composition of any one of embodiments 1-15, further comprising at least one metal chelator selected from the group consisting of ethylenediaminetetraacetic acid (EDTA) and pharmaceutically acceptable salts thereof.

Embodiment 16: The pharmaceutical composition of embodiment 15, wherein the pharmaceutically acceptable salt of ethylenediaminetetraacetic acid is disodium edetate.

Embodiment 17: The pharmaceutical composition of any one of embodiments 1-16, wherein the pharmaceutical composition is substantially free of preservatives.

Embodiment 18: The pharmaceutical composition of any one of embodiments 1-17, wherein the pharmaceutical composition is substantially free of sulfites.

Embodiment 19: A pharmaceutical composition formulated to be suitable for administration by topical administration, comprising:
phenylephrine, or a pharmaceutically acceptable salt thereof;
tropicamide, or a pharmaceutically acceptable salt thereof;
ketorolac, or a pharmaceutically acceptable salt thereof;
one or more of a poly(oxyethylene-co-oxypropylene) block copolymer,
hydroxypropyl methylcellulose, and sodium alginate; and
a pharmaceutically acceptable aqueous carrier;
wherein the pharmaceutical composition is substantially free of preservatives and substantially free of sulfites.

Embodiment 20: The pharmaceutical composition of embodiment 19, further comprising one or more of:
moxifloxacin, or a pharmaceutically acceptable salt thereof;
EDTA, or a pharmaceutically acceptable salt thereof;
boric acid,
glycerin;
polysorbate 80;
sodium metabisulfite; and
benzalkonium chloride.

Embodiment 21: The pharmaceutical composition of embodiment 19 or embodiment 20, comprising:
about 1.8 to about 2.8 wt % phenylephrine, or a pharmaceutically acceptable salt thereof;
about 0.4 to about 1.4 wt % tropicamide;
about 0.1 to about 0.8 wt % ketorolac, or a pharmaceutically acceptable salt thereof;
about 0.1 to about 0.4 wt % of a poly(oxyethylene-co-oxypropylene) block copolymer;
about 0.1 to about 0.4 wt % hydroxypropyl methylcellulose;
about 0.1 to about 0.4 wt % sodium alginate; and
a pharmaceutically acceptable aqueous carrier.

Embodiment 22: The pharmaceutical composition of embodiment 21, further comprising:
about 0.1 to about 1.0 wt % moxifloxacin, or a pharmaceutically acceptable salt thereof;
about 0.05 to about 0.15 wt % EDTA, or a pharmaceutically acceptable salt thereof;
about 0.1 to about 0.8 wt % boric acid;
about 0.25 to about 1.75 wt % glycerin;
about 0.05 to about 0.5 wt % polysorbate 80;
less than or equal to about 0.1 wt % benzalkonium chloride; and
less than or equal to about 0.15 wt % sodium metabisulfite.

Embodiment 23: The pharmaceutical composition of any one of embodiments 19-22, comprising:
about 2.3 wt % phenylephrine, or a pharmaceutically acceptable salt thereof;
about 0.9 wt % tropicamide;
about 0.37 wt % ketorolac, or a pharmaceutically acceptable salt thereof;
about 0.4 wt % of a poly(oxyethylene-co-oxypropylene) block copolymer;
about 0.23 wt % hydroxypropyl methylcellulose;
about 0.23 wt % sodium alginate; and
a pharmaceutically acceptable aqueous carrier.

Embodiment 24: The pharmaceutical composition of embodiment 23, further comprising:
about 0.51 wt % moxifloxacin, or a pharmaceutically acceptable salt thereof;
about 0.09 wt % EDTA, or a pharmaceutically acceptable salt thereof;
about 0.3 wt % boric acid;
about 1.2 wt % glycerin;
about 0.2 wt % polysorbate 80;
less than or equal to about 0.05 wt % benzalkonium chloride; and
less than or equal to about 0.1 wt % sodium metabisulfite.

Embodiment 25: A pharmaceutical composition formulated to be suitable for administration by intraocular injection, comprising:
phenylephrine, or a pharmaceutically acceptable salt thereof;
tropicamide, or a pharmaceutically acceptable salt thereof;
diclofenac, or a pharmaceutically acceptable salt thereof; and
a pharmaceutically acceptable aqueous carrier;
wherein the pharmaceutical composition is substantially free of preservatives and substantially free of sulfites.

Embodiment 26: The pharmaceutical composition of embodiment 25, further comprising one or more of: lidocaine, or a pharmaceutically acceptable salt thereof;
EDTA, or a pharmaceutically acceptable salt thereof;
boric acid,
polysorbate 80; and
sodium chloride.

Embodiment 27: The pharmaceutical composition of embodiment 25 or embodiment 26, comprising:
about 0.1 to about 0.5 wt % phenylephrine, or a pharmaceutically acceptable salt thereof;
about 0.01 to about 0.03 wt % tropicamide;
about 0.007 to about 0.013 wt % diclofenac, or a pharmaceutically acceptable salt thereof;
a pharmaceutically acceptable aqueous carrier.

Embodiment 28: The pharmaceutical composition of embodiment 27, further comprising:
about 0.1 to about 2.5 wt % lidocaine;
about 0.05 to about 0.15 wt % EDTA, or a pharmaceutically acceptable salt thereof;
about 0.1 to about 0.8 wt % boric acid;
about 0.05 to about 0.5 wt % polysorbate 80; and
about 0.1 to about 0.8 wt % sodium chloride.

Embodiment 29: The pharmaceutical composition of any one of embodiments 25-28, comprising:
about 0.29 wt % phenylephrine, or a pharmaceutically acceptable salt thereof;
about 0.02 wt % tropicamide;
about 0.01 wt % diclofenac, or a pharmaceutically acceptable salt thereof; and
a pharmaceutically acceptable aqueous carrier.

Embodiment 30: The pharmaceutical composition of embodiment 29, further comprising:
about 0.98 wt % lidocaine;
about 0.1 wt % EDTA, or a pharmaceutically acceptable salt thereof;
about 0.3 wt % boric acid;
about 0.1 wt % polysorbate 80; and
about 0.38 wt % sodium chloride.

Embodiment 31: The pharmaceutical composition of any one of embodiments 25-30, wherein the pharmaceutical composition is preservative free.

Embodiment 32: The pharmaceutical composition of any one of embodiments 25-31, wherein the pharmaceutical composition is sulfite free.

Embodiment 33: A method for surgically treating an ophthalmological disease, condition, disorder, syndrome or pathology in a mammalian subject in need of such treatment, the method comprising administering to the subject, during or prior to the surgical procedure, a pharmaceutically effective quantity of a pharmaceutical composition of any one of embodiments 1-32.

Embodiment 34: The method of embodiment 33, wherein the ophthalmological disease, condition, disorder, syndrome or pathology is selected from the group consisting of cataract, glaucoma, diseases of retina, and floppy iris syndrome.

Embodiment 35: The method of embodiment 34 wherein the gel forming compound is absent and the administration of the composition is by intraocular injection.

Embodiment 36: The method of embodiment 34, wherein the gel forming compound is present and the administration of the composition is topical.

### EXAMPLES

The following examples are provided to further elucidate the advantages and features of the present application but are not intended to limit the scope of the application. The examples are for the illustrative purposes only. USP pharmaceutical grade products were used in preparing the formulations described below.

### Example 1. Preparing a Pharmaceutical Composition #1

A pharmaceutical composition was prepared as described below. The following products were used in the amounts and concentrations specified:
(a) about 2.500 g of phenylephrine hydrochloride;
(b) about 1.000 g of tropicamide;
(c) about 0.400 g of ketorolac tromethamine;
(d) about 0.545 g of moxifloxacin hydrochloride monohydrate;
(e) about 0.100 g of edetate disodium;
( ) about 0.352 g of powdered boric acid;
(a) about 0.100 g of granulated sodium metabisulfite;
(b) about 0.200 g of POLOXAMER® 407;
(c) about 0.250 g of METHOCEL® E4M;
(d) about 0.250 g of powdered sodium alginate;
(e) about 1.000 mL of glycerin;
(1) about 0.200 mL of polysorbate 80;
(m) about 0.500 mL of 1% aqueous solution of benzalkonium chloride;
(n) about 100.0 mL of water sterile for injection; and
(o) a small quantity of 20% aqueous solution of sodium hydroxide (for adjusting pH).

To a calibrated beaker containing about 75.0 mL of water sterile for injection the entire quantities of the following five ingredients were added in the following order: (1) edetate disodium; (2) powdered boric acid; (3) phenylephrine hydrochloride; (4) ketorolac tromethamine; and (5) tropicamide. This blend was stirred until the five components were completely dissolved followed by addition of moxifloxacin hydrochloride monohydrate, with continued stirring until dissolved. POLYSORBATE® 80 and glycerin were then added and stirred, again until fully dissolved.

Then, POLOXAMER® 407, METHOCEL® E4M, and powdered sodium alginate were then added to the beaker, with continued stirring until dissolved completely, followed by addition of benzalkonium chloride and granulated sodium metabisulfite and stirring again until completely dissolved.

The pH of the final mixture was then adjusted to about 6.0 to 6.3 range using the aqueous solution of sodium hydroxide followed by adding the unused sterile water q.s., filtering through a 0.22 micron filter into sterile dropper bottles, and packaging into 11 ml Andler bottles (5 mL, 0.2 mL overfill).

### Example 2. Preparing a Pharmaceutical Composition #2

A pharmaceutical composition was prepared as described below. The following products were used in the amounts and concentrations specified:
(a) about 0.300 g of phenylephrine hydrochloride;
(b) about 0.020 g of tropicamide;
(c) about 1.000 g of lidocaine;
(d) about 0.010 g of diclofenac sodium;
(e) about 0.100 g of edetate disodium;
(f) about 0.352 g of powdered boric acid;
(g) about 0.386 g of granulated sodium chloride;
(h) about 0.100 mL of polysorbate 80;
(i) about 100.0 mL of water sterile for injection; and
(j) a small quantity of 10% aqueous solution of sodium hydroxide (for adjusting pH).

To a calibrated beaker containing about 80.0 mL of water sterile for injection the entire quantities of the following six ingredients were added in the following order: (1) sodium chloride; (2) edetate disodium; (3) powdered boric acid; (4) lidocaine; (5) phenylephrine hydrochloride; and (6) tropicamide. This blend was stirred until the five components were completely dissolved, slightly heating the blend to not more than 30° C. followed by addition of POLYSORBATE ° 80, with continued stirring until dissolved. Finally, diclofenac was added and stirred, again until fully dissolved.

The pH of the final mixture was then adjusted to about 6.5±0.1 using the aqueous solution of sodium hydroxide followed by adding the unused sterile water q.s., filtering through a 0.22 micron filter into sterile dropper bottles, and packaging into 11 ml Andler bottles (5 mL, 0.2 mL overfill), capping, sealing, and labeling.

Although the invention has been described with reference to the above examples, it will be understood that modifications and variations are encompassed within the spirit and scope of the invention. Accordingly, the invention is limited only by the following claims.

### Example 3. Assessment of the Stability of Pharmaceutical Composition #1

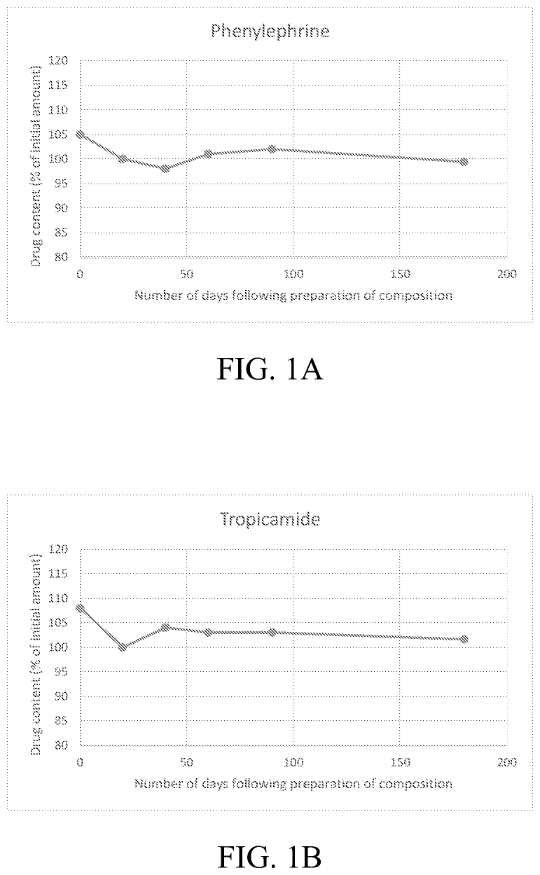

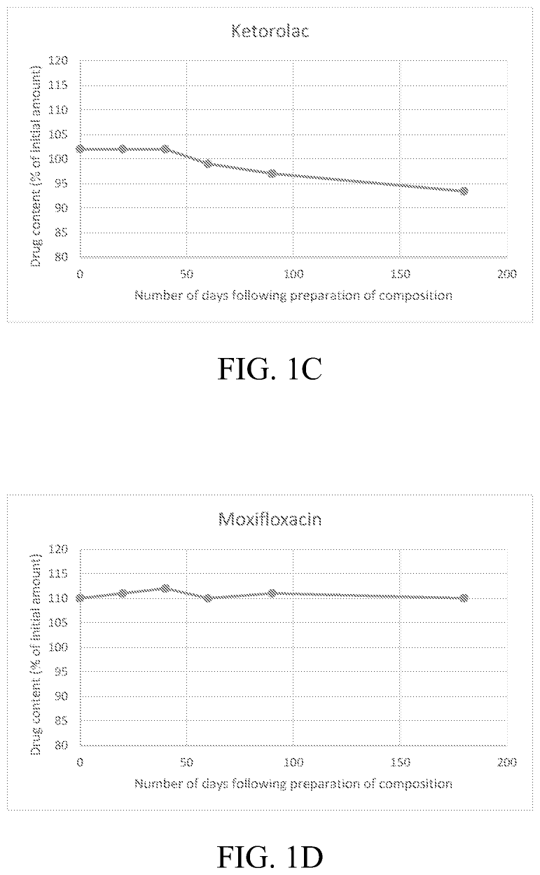

The pharmaceutical composition described in Example 1 was stored at room temperature for a six-month period. At the time the compositions were first prepared and then following 20 days, 40 days, 60 days, 90 days, and 180 days of storage, the pharmaceutical composition was analyzed via high performance liquid chromatography (HPLC) to determine the content of each active ingredient in the composition (phenylephrine, tropicamide, ketorolac, and moxifloxacin). As depicted in FIG. 1, the amount of each active ingredient remained close to the initial value over the course of the study, indicating an optimal stability profile for the composition. These data validate the ability of the composition to be stored at room temperature for an extended period of time without degradation of any of the active ingredients.

### Example 4. Assessment of the Stability of Pharmaceutical Composition #2

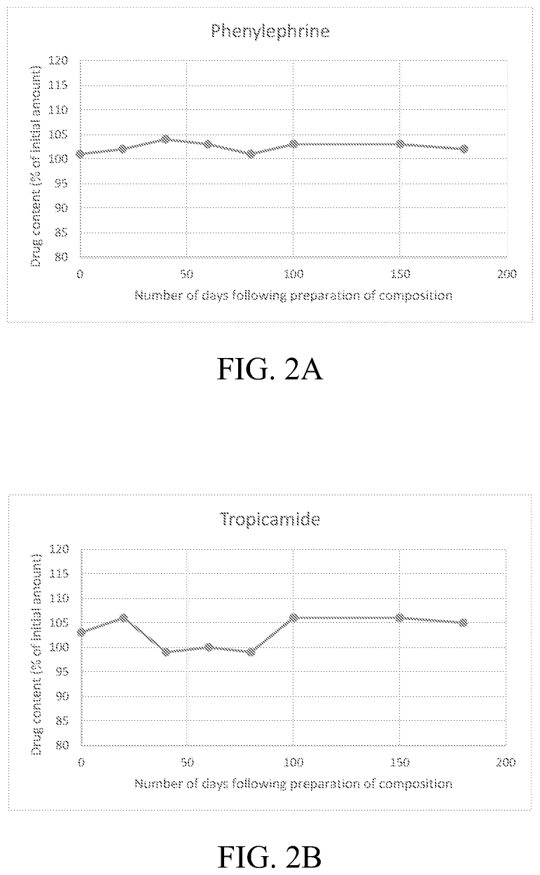

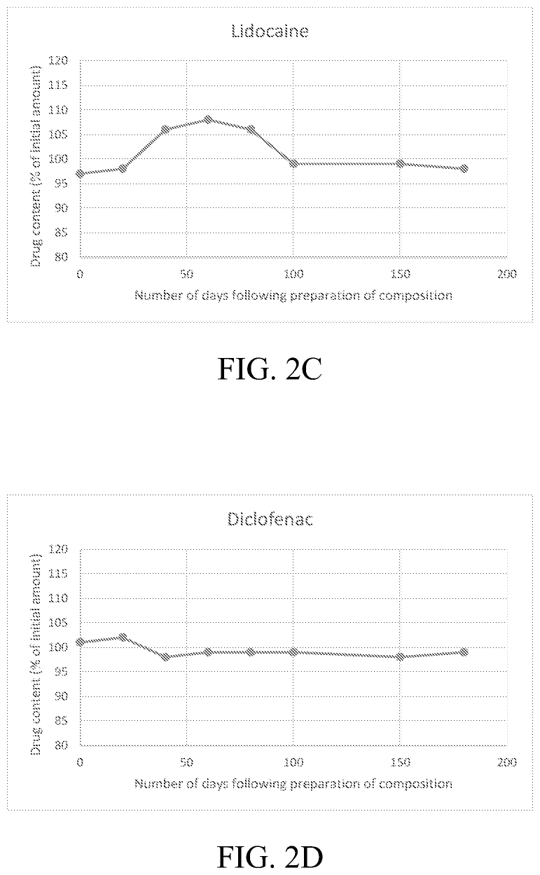

The pharmaceutical composition described in Example 2 was stored at room temperature for a six-month period. At the time the compositions were first prepared and then following 20 days, 40 days, 60 days, 80 days, 100 days, 150 days, and 180 days of storage, the pharmaceutical composition was analyzed via high performance liquid chromatography (HPLC) to determine the content of each active ingredient in the composition (phenylephrine, tropicamide, lidocaine, and diclofenac). As depicted in FIG. 2, the amount of each active ingredient remained close to the initial value over the course of the study, indicating an optimal stability profile for the composition. These data validate the ability of the composition to be stored at room temperature for an extended period of time without degradation of any of the active ingredients.

1. A pharmaceutical composition, comprising:
(a) a therapeutically effective quantity of two mydriatic compounds;
(b) a therapeutically effective quantity of at least one non-steroid anti-inflammatory drug;
(c) optionally, a quantity of a one or more gel forming compounds;
(d) a pharmaceutically acceptable aqueous carrier,
wherein the pharmaceutical composition is optionally free of preservatives and is optionally free of sulfites, with the further proviso that the pharmaceutical composition is formulated to be suitable for administration by an intraocular injection or for topical administration.

2. The pharmaceutical composition of claim 1, wherein the two mydriatic compounds are selected from the group consisting of phenylephrine, tropicamide, brimonidine, cyclopentolate, cyclopentolate hydrochloride, atropine, homatropine, scopolamine; and pharmaceutically acceptable salts thereof.

3. The pharmaceutical composition of claim 1, wherein the two mydriatic compounds are phenylephrine and tropicamide, or pharmaceutically acceptable salts thereof.

4. The pharmaceutical composition claim 1, wherein the non-steroid anti-inflammatory drug is selected from the group consisting of ketorolac, bromfenac, etodolac, sulindac, diclofenac, aceclofenac, nepafenac, tolmetin, indomethacin, nabumetone, ketoprofen, dexketoprofen, ibuprofen, flurbiprofen, dexibuprofen, fenoprofen, loxoprofen, oxaprozin, naproxen, aspirin, salicylic acid, diflunisal, salsalate, mefenamic acid, meclofenamic acid, flufenamic acid, tolfenamic acid, meloxicam, piroxicam, ternoxicam, droxicam, lornoxicam, isoxicam, celecoxib, rofecoxib, valdecoxib, parecoxib, lumiracoxib, etoricoxib, firocoxib, nimesulide, clonixin, licofelone, and pharmaceutically acceptable salts thereof.

5. The pharmaceutical composition of claim 1, wherein the non-steroid anti-inflammatory drug is ketorolac, or a pharmaceutically acceptable salt thereof.

6. The pharmaceutical composition of claim 1, wherein the non-steroid anti-inflammatory drug is diclofenac, or a pharmaceutically acceptable salt thereof.

7. The pharmaceutical composition of claim 1, wherein the one or more gel forming compounds is selected from the group consisting of alginic acid, sodium alginate, potassium alginate, calcium alginate, agar-agar, pectin, guar gum, xanthan gum, gelatin, poly(oxyethylene-co-oxypropylene) block copolymers, poly(N-isopropylacrylamide), poly(N-i sopropylacrylamide-co-acrylic acid), poly(vinyl pyrrolidone), poly(-vinylpyridine-co-ethylacrylate) block copolymers, poly(N-isopropylacrylamide-co-butyl methacrylate-co-ethylene glycol) block copolymers, carboxymethyl cellulose hydroxyethyl cellulose, methyl cellulose, hydroxypropyl cellulose, hydroxypropyl methylcellulose, polyoxyethylene sorbitan monolaurates, polyoxyethylene sorbitan monopalmitates, polyoxyethylene sorbitan monostearates, and polyoxyethylene sorbitan monooleates.

8. The pharmaceutical composition of claim 1, wherein the one or more gel forming compounds comprises sodium alginate.

9. The pharmaceutical composition of claim 1, wherein the one or more gel forming compounds comprises a quantity of a poly(oxyethylene-co-oxypropylene) block copolymer.

10. The pharmaceutical composition of claim 1, wherein the one or more gel forming compounds comprises hydroxypropyl methylcellulose.

11. The pharmaceutical composition of claim 1, further comprising a therapeutically effective quantity of at least one anesthetic compound selected from the group consisting of lidocaine, tetracaine, proparacaine, procaine, dyclonine, chloroprocaine, and pharmaceutically acceptable salts thereof.

12. The pharmaceutical composition of claim 11, wherein the anesthetic compound is lidocaine, or a pharmaceutically acceptable salt thereof.

13. The pharmaceutical composition of claim 1, further comprising a therapeutically effective quantity of at least one antibiotic selected from the group consisting of moxifloxacin, gatifloxacin, teicoplanin, telavancin, decaplanin, ramoplanin ciprofloxacin, besifloxacin, levofloxacin, gentamicin, tobramycin and amikacin, and pharmaceutically acceptable salts thereof.

14. The pharmaceutical composition of claim 13, wherein the antibiotic is moxifloxacin, or a pharmaceutically acceptable salt thereof.

15. The pharmaceutical composition of claim 1, further comprising at least one metal chelator selected from the group consisting of ethylenediaminetetraacetic acid (EDTA) and pharmaceutically acceptable salts thereof.

16. The pharmaceutical composition of claim 15, wherein the pharmaceutically acceptable salt of ethylenediaminetetraacetic acid is disodium edetate.

17. The pharmaceutical composition of claim 1, wherein the pharmaceutical composition is substantially free of preservatives.

18. The pharmaceutical composition of claim 1, wherein the pharmaceutical composition is substantially free of sulfites.

19. A pharmaceutical composition formulated to be suitable for administration by topical administration, comprising:
phenylephrine, or a pharmaceutically acceptable salt thereof;
tropicamide, or a pharmaceutically acceptable salt thereof;
ketorolac, or a pharmaceutically acceptable salt thereof;
one or more of a poly(oxyethylene-co-oxypropylene) block copolymer,
hydroxypropyl methylcellulose, and sodium alginate; and
a pharmaceutically acceptable aqueous carrier;
wherein the pharmaceutical composition is substantially free of preservatives and substantially free of sulfites.

20-24. (canceled)

25. A pharmaceutical composition formulated to be suitable for administration by
intraocular injection, comprising:
phenylephrine, or a pharmaceutically acceptable salt thereof;
tropicamide, or a pharmaceutically acceptable salt thereof;
diclofenac, or a pharmaceutically acceptable salt thereof; and
a pharmaceutically acceptable aqueous carrier;
wherein the pharmaceutical composition is substantially free of preservatives and substantially free of sulfites.

26-36. (canceled)
